(19) United States
(12) Patent Application Publication
(10) Pub. No.: US 2026/0232800 A1
(43) Pub. Date: Aug. 13, 2026

(54) T CELL RECEPTOR

(71) Applicant: Adaptimmune Limited, Abingdon, Oxfordshire (GB)

(72) Inventors: William Lawrance, Abingdon, Oxfordshire (GB); Philip Clive Simister, Abingdon, Oxfordshire (GB)

(21) Appl. No.: 19/138,071

(22) PCT Filed: Dec. 13, 2023
(86) PCT No.: PCT/GB2023/053213

§ 371 Date: Jun. 11, 2025

(30) Foreign Application Priority Data
Dec. 13, 2022 (GB) 2218754.6

(51) Int. Cl. A61K 40/42; A61K 38/00; A61K 40/11; A61K 40/32; A61K 47/64; A61K 49/00; C07K 14/705; C12N 5/0783

(52) U.S. Cl. CPC A61K 40/4202; A61K 40/11; A61K 40/32; A61K 47/6425; A61K 49/0002; C07K 14/7051; C12N 5/0636; A61K 38/00; C12N 2510/00

(57) ABSTRACT

The disclosure relates to T cell receptors (TCRs) that can bind to HLA-A*02 restricted peptides derived from PRAME. The disclosure also relates to complexes comprising these TCRs, nucleic acids and vectors encoding these TCRs, or portions thereof, cells comprising these TCRs, pharmaceutical compositions, and methods of using these TCRs to treat diseases, such as cancer.

### FIELD OF THE DISCLOSURE

The disclosure relates to T cell receptors (TCRs) that can bind to HLA-A*02 restricted peptides derived from PRAME. The disclosure also relates to complexes comprising these TCRs, nucleic acids and vectors encoding these TCRs, or portions thereof, cells comprising these TCRs, pharmaceutical compositions, and methods of using these TCRs to treat diseases, such as cancer.

### BACKGROUND

T cell receptors (TCRs) are naturally expressed by CD4+ and CD8+ T cells. TCRs recognize short peptide antigens that are displayed on the surface of antigen presenting cells in complex with Major Histocompatibility Complex (MHC) molecules. In humans, MHC molecules are also known as Human Leukocyte Antigens, or HLAs (Davis et al., Annu Rev Immunol. 1998; 16:523-44). CD8+ T cells, which are also termed cytotoxic T cells, have TCRs that specifically recognize peptides bound to MHC class I molecules. CD8+ T cells are generally responsible for finding and mediating the destruction of diseased cells, such as cancerous and virally infected cells. The affinity of cancer-specific TCRs in the natural repertoire for their corresponding antigen is typically low as a result of thymic selection, meaning that cancerous cells frequently escape detection and destruction by T cells. Novel immunotherapeutic approaches aimed at promoting cancer recognition by T cells offer a highly promising strategy for the development of effective anti-cancer treatments.

PRAME or Preferentially Expressed Antigen In Melanoma was first identified as an antigen that is over expressed in melanoma (Ikeda et al Immunity. 1997 February; 6(2): 199-208). It has since been found to be over expressed in other forms of cancer, such as leukaemia, lymphoma, lung cancer, breast cancer, ovarian cancer, endometrial cancer, oesophageal cancer, bladder cancer and head and neck cancers (Doolan et al Breast Cancer Res Treat. 2008 May; 109(2): 359-65; Epping et al Cancer Res. 2006 Nov. 15; 66(22): 10639-42; Ercolak et al Acta Haematol. 2015; 134(4): 199-207; Matsushita et al Leuk Lymphoma. 2003 March; 44(3): 439-44; Mitsuhashi et al Int. J Hematol. 2014; 100(1): 88-95; Proto-Siqueira et al Leuk Res. 2006 November; 30 (11): 1333-9; Szczepanski et al Oral Oncol. 2013 February; 49(2): 144-51; Van Baren et al Br J Haematol. 1998 September; 102(5): 1376-9).

PRAME is also known in the literature as CT130, MAPE, and OIP-4. The polypeptide sequence of human PRAME can be found at Uniprot accession number P78395. PRAME functions as a repressor of retinoic acid receptor signalling (Epping et al., Cell. 2005 Sep. 23; 122(6):835-47) and belongs to the family of germline-encoded antigens known as cancer testis antigens. Cancer testis antigens are a family of tumour-associated antigens expressed in human tumours of different histological origin, but not in normal human tissues except for testis and placenta. Cancer testis antigens therefore represent attractive targets for immunotherapeutic intervention since they have limited or no expression in normal adult tissues. High affinity TCRs for cancer testis antigens are thus desired.

### SUMMARY OF THE DISCLOSURE

Provided herein are TCRs that can bind to a complex comprising PRAME-derived peptides and HLA-A*02. These TCR are highly advantageous, for example, in the treatment of PRAME-expressing cancers.

In more detail, the present inventors have identified advantageous TCRs that bind to a complex comprising SLLQHLIGL (SEQ ID NO: 1) and HLA-A*02. The peptide SLLQHLIGL (SEQ ID NO: 1) corresponds to amino acids 425-433 of the full length PRAME protein (Uniprot accession number P78395) and is presented on the cell surface in complex with HLA-A*02 (Kessler et al., J Exp Med. 2001 Jan. 1; 193(1):73-88). The TCRs of the present disclosure have been engineered to bind the target antigen with increased affinity relative to any naturally-occurring TCRs. As such the TCRs of the present disclosure are particularly advantageous, for example, in therapeutic applications such as the treatment of cancer.

Accordingly, the disclosure provides a TCR that binds to a complex comprising SEQ ID NO: 1 and HLA-A*02, and comprises: (a) a TCR alpha chain variable domain that comprises an amino acid sequence that has at least 50% sequence identity to SEQ ID NO: 17; and (b) a TCR beta chain variable domain that comprises an amino acid sequence that has at least 50% sequence identity to SEQ ID NO: 29.

The disclosure also provides a complex comprising (a) a TCR of the disclosure, and (b) a detectable label, a therapeutic agent or a pharmacokinetic modifying moiety. The disclosure also provides a nucleic acid encoding (a) a TCR of the disclosure; (b) a TCR alpha chain variable domain of a TCR of the disclosure, and optionally a TCR alpha chain constant domain; or (c) a TCR beta chain variable domain of a TCR of the disclosure, and optionally a TCR beta chain constant domain. The disclosure also provides a vector comprising a nucleic acid of the disclosure.

The disclosure also provides a virus particle comprising a nucleic acid of the disclosure or a vector of the disclosure. The disclosure also provides a cell that: (a) expresses a TCR of the disclosure; (b) comprises at least one nucleic acid of the disclosure; optionally wherein the cell comprises (i) a first nucleic acid that encodes a TCR alpha chain variable domain of a TCR of the disclosure and optionally a TCR alpha chain constant domain, and (ii) a second nucleic acid that encodes a TCR beta chain variable domain of a TCR of the disclosure and optionally a TCR beta chain constant domain; or (c) comprises at least one vector of the disclosure; optionally wherein the cell comprises (i) a first vector that comprises a nucleic acid that encodes a TCR alpha chain variable domain of a TCR of the disclosure and optionally a TCR alpha chain constant domain, and (ii) a second vector that comprises a nucleic acid that encodes a TCR beta chain variable domain of a TCR of the disclosure and optionally a TCR beta chain constant domain. The disclosure also provides a pharmaceutical composition comprising (i) a TCR of the disclosure, a complex of the disclosure, a nucleic acid of the disclosure, or a cell of the disclosure, and (ii) one or more pharmaceutically acceptable carriers or excipients.

The disclosure also provides a TCR of the disclosure, a nucleic acid of the disclosure, a cell of the disclosure, or a pharmaceutical composition of the disclosure for use in a method of treating a disease in an individual, the method comprising administering the TCR, the nucleic acid, the cell or the pharmaceutical composition to the individual. Similarly, the disclosure provides a method of treating a disease in an individual, comprising administering to the individual a TCR of the disclosure, a nucleic acid of the disclosure, a cell of the disclosure, or a pharmaceutical composition of the disclosure.

The disclosure also provides a method of making one or more cell of the disclosure comprising: (i) introducing to a cell at least one nucleic acid of the disclosure or at least one vector of the disclosure; and then optionally (ii) culturing the cell such that the cell proliferates to form a population.

### BRIEF DESCRIPTION OF THE FIGURES

FIG. 1: Increasing functional avidity of potency-enhanced TCRs specific for HLA-A*02:01 PRAME_SLLQHLIGL. A panel of TCRs transduced into Jurkat cells were co-cultured for 16 hours at a 1:1 ratio with T2 cells pulsed with 10-fold dilutions of PRAME_SLLQHLIGL peptide from 1×10⁻⁵ M down to 1×10⁻¹² M. Increases in CD69 responses measured by flow cytometry are apparent from the titration curves (left graph), with EC50 values ranging from 14 to 0.1 nM, a 2-log spread. EC₅₀ values indicate the peptide concentration required to induce half-maximal Jurkat cell activation (% CD69⁺ cells). Titrated peptide concentrations are labelled (graph x-axis) as log values, and EC50 values are listed equivalently along with corresponding values converted to nM (in the table, right).

FIG. 2: TCR-transduced T-cell panel shows specificity for PRAME as measured by IFNγ ELISPot. A panel of six engineered TCR-transduced donor T-cells was investigated by IFNγ ELISpot assay in the presence of 4 PRAME-positive tumour cell lines (Malme-3M, SK-Mel-5, NCI-H1755, NCI-H1703) and 2 PRAME-negative (or negligible) tumour cell lines (NCI-H2228, TCC-SUP). Non-transduced T-cells (NTD) were included with each target cell line as were the target cells alone, ‘Targets only’ (both with responses mostly too low to see). The measured spot counts after IFNγ release are shown. The data highlight the target specificity of all engineered TCR T-cells (dark to medium grey bars), which is consistent with tumour cell line RNA expression level (Malme-3M≥SK-Mel-5>NCI-H1755>NCI-H1703). The higher PRAME-expressing cell lines (Malme-3M, SK-Mel-5, and NCI-H1755) elicited strong T-cell responses across the PRAME TCR T-cell panel, which responded variably and with lower signals to a lower PRAME-expressing cell line (NCI-H1703). Negligible or no T-cell activation responses were measured on PRAME-negative cell lines (NCI-H2228, TCC-SUP), and no or negligible activation responses were observed with all NTD T-cells, or target cells only (lightest shades of grey). Note, maximum response signals provide a semi-quantitative readout as counts may be above the maximum countable signal (500 spots). Values are means of duplicates, with error bars showing the signal range. The boxed key (bottom) indicates the grey-shading colour scheme of each plotted effector T-cell type, or no added T-cell (Targets only).

FIG. 3: Cytotoxicity of selected TCR-transduced T-cells against a PRAME-positive tumour cell line (Malme-3M). 2D killing responses to target Malme-3M tumour (melanoma) cell line when co-cultured with either non-transduced (ntd) T-cells, or T-cells transduced with selected PRAME-specific TCRs (ADB02780_063, _070, _077) are shown in the upper row (main panel, left). Potent target-cell killing is observed within 24 hours after co-culturing with each PRAME-specific TCR T-cell; cytotoxicity continued for at least 3 further days. No killing was observed with ntd T-cells lacking a PRAME TCR (upper row, leftmost graph). In the lower row (main panel), corresponding ntd or transduced effector T-cells were incubated alone without Malme-3M target cells, and no killing responses observed. Malme-3M target cells alone, with no added effector T-cells, also show no cell killing (upper right, small panel). The assay monitored cell apoptotic activity over time (in hours) in an IncuCyte ZOOM live cell imaging system. The responses (Green Object Count) represent apoptotic cell number measured/mm². Data for all co-cultured T-cells and T-cells alone (main panel, left) are shown as mean±/−SEM of 3 replicates. Malme-3M targets alone data are single replicates. Plots are limited to 96 hours after T-cell addition.

FIG. 4: Functional avidity of potency-enhanced TCRs specific for PRAME_SLLQHLIGL presented by HLA-A*02:01. A panel of TCRs transduced into primary T-cells were co-cultured for 48 hours at a 1:1 ratio with T2 target cells (PRAME negative, HLA-A*02 positive) pulsed with 10-fold dilutions of PRAME_SLLQHLIGL peptide from 1×10⁻⁵ M down to 1×10⁻¹¹ M. Potency was measured by IFNγ supernatant ELISA. EC₅₀ values indicate the peptide concentration required to induce half-maximal T-cell activation with values ranging from −10-8.8 M to 10-7.8 M across the panel of mutants.

FIG. 5: T-cells transduced to express potency-enhanced TCRs demonstrate specificity for PRAME when tested against a panel of PRAME-positive and -negative cell lines. Target cells were incubated with the indicated TCR transduced T-cells alone (closed circles) or in the presence of exogenous 10⁻⁵ M PRAME peptide, SLLQHLIGL (open circles, ADB02780_070 only) for ~42 hours, and IFNγ in the supernatant was measured by ELISA. T-cells derived from 4 donors were tested (DVA_002, GPA_025, KXA_039 and XKA_003, presented as separate panels). The bottom panel provides the target tumour cell lines, with transcript level (as determined by qPCR, normalised to reference gene levels) shown in ascending order from left to right. Non-transduced T-cells (ntd) and targets alone conditions were included as negative controls. The top panel provides the IFNγ ELISA results.

FIG. 6: T-cells transduced to express potency-enhanced TCRs demonstrate specificity for PRAME when tested against a panel of antigen-negative primary cell lines. The potency of 3 engineered TCRs towards a panel of antigen-negative (HLA*02-positive) primary cell lines was investigated using IFNγ supernatant ELISA. Target cells were incubated with TCR transduced T-cells alone (closed circles) or in the presence of exogenous 10-5 M PRAME_SLLQHLIGL peptide (open circles, ADB02780_070 only) for ~42 hours. T-cells derived from 4 donors were tested (DVA_002, GPA_025, KXA_039 and XKA_003, presented as separate panels). Primary cell lines are ranked according to transcript level (as determined by qPCR) in ascending order from left to right. Non-transduced T-cells (ntd) and targets alone conditions were included as negative controls.

### DETAILED DESCRIPTION

It is to be understood that different applications of the disclosed products and methods may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a TCR” includes “TCRs”, reference to “an antibody” includes two or more such antibodies, reference to “a cell” includes two or more such cells, and the like.

In general, the term “comprising” is intended to be interpreted as being open-ended terms (i.e., meaning “including, but not limited to”), unless otherwise noted.

In some aspects of the disclosure, the word “comprising” is replaced with the phrase “consisting of”. The term “consisting of” is intended to be limiting.

The terms “cancer,” “neoplasm,” and “tumour” are used interchangeably and, in either the singular or plural form, refer to cells that have undergone a malignant transformation that makes them pathological to the host organism.

The terms “protein” and “polypeptide” are used interchangeably herein, and are intended to refer to a polymeric chain of amino acids of any length.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm.

The term “T-cell receptor” or “TCR” refers to a heterodimeric molecule comprising an alpha polypeptide chain (alpha chain) and a beta polypeptide chain (beta chain), wherein the heterodimeric receptor is capable of binding to a peptide antigen presented by an MHC molecule. TCRs are described herein using the International Immunogenetics (IMGT) TCR nomenclature, and links to the IMGT public database of TCR sequences. Native alpha-beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number. Thus “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV5-1” defines a TCR Vβ region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region. The alpha and beta chains of αβ TCRs are generally regarded as each having two “domains”, namely variable and constant domains. The variable domain consists of a concatenation of variable region and joining region. In the process of joining the TRAV and TRAJ gene segments together, a few DNA bases are randomly added or deleted. This helps generate diversity in CDR3 sequences, with each T cell generating a unique TCR alpha chain gene. Likewise, in the process of joining the TRBV and TRBJ gene segments together, a few DNA bases are randomly added or deleted, such that diversity is provided in CDR3 sequences and each T cell generates a unique TCR beta chain gene. Thus, in the present specification and claims, the term “TCR alpha variable domain” refers to the concatenation of TRAV and TRAJ regions, and the term TCR alpha constant domain refers to the extracellular TRAC region, or to a C-terminal truncated TRAC sequence. Likewise the term “TCR beta variable domain” refers to the concatenation of TRBV and TRBD/TRBJ regions, and the term TCR beta constant domain refers to the extracellular TRBC region, or to a C-terminal truncated TRBC sequence.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

### T Cell Receptor (TCR)

The TCR of the disclosure is capable of binding a complex comprising the PRAME peptide sequence SLLQHLIGL (SEQ ID NO: 1) and HLA-A*02. The TCR of the disclosure comprises a TCR alpha chain variable domain that comprises an amino acid sequence that has at least 50% sequence identity to SEQ ID NO: 17; and (b) a TCR beta chain variable domain that comprises an amino acid sequence that has at least 50% sequence identity to SEQ ID NO: 29.

SEQ ID NO: 17 is the alpha chain variable domain sequence, and SEQ ID NO: 29 is the beta chain variable domain sequence of a parental TCR, termed TCR1. As outlined in the Examples of the present application, eight affinity matured TCRs were generated from TCR1 which are particularly efficacious, termed TCR2-9. Table 1 below summarises the sequences of each of the TCRs 2-9.

TABLE 1
SEQ ID Nos of TCRs. TCR numbers (1 to 9) and ADB references (ADB02780_001,
ADB02780_022 etc.) may be used interchangeably in the disclosure.

| | Alpha chain | | | | Beta chain | | | |
|---|---|---|---|---|---|---|---|---|
| TCR no. | CDR1 | CDR2 | CDR3 | Variable | CDR1 | CDR2 | CDR3 | Variable |
| 1 | 8 | 15 | 16 | 17 | 24 | 25 | 28 | 29 |
| (ADB02780_001) | | | | | | | | |
| 2 | 8 | 15 | 16 | 17 | 24 | 26 | 28 | 30 |
| (ADB02780_022) | | | | | | | | |
| 3 | 8 | 15 | 16 | 17 | 24 | 27 | 28 | 31 |
| (ADB02780_025) | | | | | | | | |
| 4 | 9 | 15 | 16 | 18 | 24 | 27 | 28 | 31 |
| (ADB02780_062) | | | | | | | | |
| 5 | 10 | 15 | 16 | 19 | 24 | 27 | 28 | 31 |
| (ADB02780_063) | | | | | | | | |
| 6 | 11 | 15 | 16 | 20 | 24 | 27 | 28 | 31 |
| (ADB02780_070) | | | | | | | | |
| 7 | 12 | 15 | 16 | 21 | 24 | 27 | 28 | 31 |
| (ADB02780_077) | | | | | | | | |
| 8 | 13 | 15 | 16 | 22 | 24 | 27 | 28 | 31 |
| (ADB02780_106) | | | | | | | | |
| 9 | 14 | 15 | 16 | 23 | 24 | 27 | 28 | 31 |
| (ADB02780_107) | | | | | | | | |

### Variable Domains

As set out above, the TCR of the disclosure comprises a TCR alpha chain variable domain that comprises an amino acid sequence that has at least 50% sequence identity to SEQ ID NO: 17. The TCR alpha chain variable domain may, for example, comprise an amino acid sequence that has at least 60%, at least 70%, at least 75%, at least 80%, at least 90%, or at least 95% sequence identity to SEQ ID NO: 17.

The TCR alpha chain variable domain may, for example, comprise a CDR1 that comprises (1) the sequence of SEQ ID NO: 8 or (2) an amino acid sequence that comprises one, two or three amino acid insertions, deletions or substitutions relative to the sequence of SEQ ID NO: 8. The amino acid sequence that comprises one, two or three amino acid insertions, deletions or substitutions relative to the sequence of SEQ ID NO: 8 may, for example, comprise SEQ ID NO: 9, 10, 11, 12, 13 or 14. SEQ ID NOs: 9, 10, 11, 12, 13 and 14 are single substitution mutants of SEQ ID NO: 8. The TCR alpha chain variable domain may, for example, comprise a CDR2 that comprises (1) the sequence of SEQ ID NO: 15 or (2) an amino acid sequence that comprises one, two or three amino acid insertions, deletions or substitutions relative to the sequence of SEQ ID NO: 15. The TCR alpha chain variable domain may, for example, comprise a CDR3 that comprises (1) the sequence of SEQ ID NO: 16 or (2) an amino acid sequence that comprises one, two or three amino acid insertions, deletions or substitutions relative to the sequence of SEQ ID NO: 16.

The TCR alpha chain variable domain may, for example, comprise: (i) a CDR1 that comprises (1) the sequence of SEQ ID NO: 8 or (2) an amino acid sequence that comprises one, two or three amino acid insertions, deletions or substitutions relative to the sequence of SEQ ID NO: 8; (ii) a CDR2 that comprises (1) the sequence of SEQ ID NO: 15 or (2) an amino acid sequence that comprises one, two or three amino acid insertions, deletions or substitutions relative to the sequence of SEQ ID NO: 15; and/or (iii) a CDR3 that comprises (1) the sequence of SEQ ID NO: 16 or (2) an amino acid sequence that comprises one, two or three amino acid insertions, deletions or substitutions relative to the sequence of SEQ ID NO: 16. For instance, the TCR alpha chain variable domain may comprise (i); (ii); (iii); (i) and (ii); (i) and (iii), (ii) and (iii); or (i), (ii) and (iii). In one aspect of the disclosure, the TCR alpha chain variable domain comprises (i), (ii) and (iii).

The TCR of the disclosure also comprises a TCR beta chain variable domain that comprises an amino acid sequence that has at least 50% sequence identity to SEQ ID NO: 29. The TCR beta chain variable domain may, for example, comprise an amino acid sequence that has at least 60%, at least 70%, at least 75%, at least 80%, at least 90%, or at least 95% sequence identity to SEQ ID NO: 29.

The TCR beta chain variable domain may, for example, comprise a CDR1 that comprises (1) the sequence of SEQ ID NO: 24 or (2) an amino acid sequence that comprises one, two or three amino acid insertions, deletions or substitutions relative to the sequence of SEQ ID NO: 24. The TCR beta chain variable domain may, for example, comprise a CDR2 that comprises (1) the sequence of SEQ ID NO: 25 or (2) an amino acid sequence that comprises one, two or three amino acid insertions, deletions or substitutions relative to the sequence of SEQ ID NO: 25. The amino acid sequence that comprises one, two or three amino acid insertions, deletions or substitutions relative to the sequence of SEQ ID NO: 25 may, for example, comprise SEQ ID NO: 26 or 27. SEQ ID NOs: 26 and 27 are single substitution mutants of SEQ ID NO: 25. The TCR beta chain variable domain may, for example, comprise a CDR3 that comprises (1) the sequence of SEQ ID NO: 28 or (2) an amino acid sequence that comprises one, two or three amino acid insertions, deletions or substitutions relative to the sequence of SEQ ID NO: 28.

The TCR beta chain variable domain may, for example, comprise: (iv) a CDR1 that comprises (1) the sequence of SEQ ID NO: 24 or (2) an amino acid sequence that comprises one, two or three amino acid insertions, deletions or substitutions relative to the sequence of SEQ ID NO: 24; (v) a CDR2 that comprises (1) the sequence of SEQ ID NO: 25 or (2) an amino acid sequence that comprises one, two or three amino acid insertions, deletions or substitutions relative to the sequence of SEQ ID NO: 25; and/or (vi) a CDR3 that comprises (1) the sequence of SEQ ID NO: 28 or (2) an amino acid sequence that comprises one, two or three amino acid insertions, deletions or substitutions relative to the sequence of SEQ ID NO: 28. For instance, the TCR beta chain variable domain may comprise (iv); (v); (vi); (iv) and (v); (iv) and (vi); (v) and (vi); or (iv), (v) and (vi). In one aspect of the disclosure, the TCR beta chain variable domain comprises (iv), (v) and (vi).

The TCR may comprise: (a) a TCR alpha chain variable domain comprising: (i) a CDR1 that comprises (1) the sequence of SEQ ID NO: 8 or (2) an amino acid sequence that comprises one, two or three amino acid insertions, deletions or substitutions relative to the sequence of SEQ ID NO: 8; (ii) a CDR2 that comprises (1) the sequence of SEQ ID NO: 15 or (2) an amino acid sequence that comprises one, two or three amino acid insertions, deletions or substitutions relative to the sequence of SEQ ID NO: 15; and/or (iii) a CDR3 that comprises (1) the sequence of SEQ ID NO: 16 or (2) an amino acid sequence that comprises one, two or three amino acid insertions, deletions or substitutions relative to the sequence of SEQ ID NO: 16; and (b) a TCR beta chain variable domain comprising: (iv) a CDR1 that comprises (1) the sequence of SEQ ID NO: 24 or (2) an amino acid sequence that comprises one, two or three amino acid insertions, deletions or substitutions relative to the sequence of SEQ ID NO: 24; (v) a CDR2 that comprises (1) the sequence of SEQ ID NO: 25 or (2) an amino acid sequence that comprises one, two or three amino acid insertions, deletions or substitutions relative to the sequence of SEQ ID NO: 25; and/or (vi) a CDR3 that comprises (1) the sequence of SEQ ID NO: 28 or (2) an amino acid sequence that comprises one, two or three amino acid insertions, deletions or substitutions relative to the sequence of SEQ ID NO: 28. For example, the TCR may comprise (i) and (iv); (i) and (v); (i) and (vi); (i), (iv) and (v); (i), (iv) and (vi); (i), (v) and (vi); (i), (iv), (v) and (vi); (ii) and (iv); (ii) and (v); (ii) and (vi); (ii), (iv) and (v); (ii), (iv) and (vi); (ii), (v) and (vi); (ii), (iv), (v) and (vi); (iii) and (iv); (iii) and (v); (iii) and (vi); (iii), (iv) and (v); (iii), (iv) and (vi); (iii), (v) and (vi); (iii), (iv), (v) and (vi); (i), (ii) and (iv); (i), (ii) and (v); (i), (ii) and (vi); (i), (ii), (iv) and (v); (i), (ii), (iv) and (vi); (i), (ii), (v) and (vi); (i), (ii), (iv), (v) and (vi); (i), (iii) and (iv); (i), (iii) and (v); (i), (iii) and (vi); (i), (iii), (iv) and (v); (i), (iii), (iv) and (vi); (i), (iii), (v) and (vi); (i), (iii), (iv), (v) and (vi); (ii), (iii) and (iv); (ii), (iii) and (v); (ii), (iii) and (vi); (ii), (iii), (iv) and (v); (ii), (iii), (iv) and (vi); (ii), (iii), (v) and (vi); (ii), (iii), (iv), (v) and (vi); (i), (ii), (iii) and (iv); (i), (ii), (iii) and (v); (i), (ii), (iii) and (vi); (i), (ii), (iii), (iv) and (v); (i), (ii), (iii), (iv) and (vi); (i), (ii), (iii), (v) and (vi); or (i), (ii), (iii), (iv), (v) and (vi). In one aspect of the disclosure, the TCR alpha chain variable domain comprises (i), (ii) and (iii) and the TCR beta chain variable domain comprises (iv), (v) and (vi).

In some cases, (a) the TCR alpha chain variable domain comprises a CDR1 that comprises SEQ ID NO: 8, 9, 10, 11, 12, 13 or 14; (b) the TCR alpha chain variable domain comprises a CDR2 that comprises SEQ ID NO: 15; (c) the TCR alpha chain variable domain comprises a CDR3 that comprises SEQ ID NO: 16; (d) the TCR beta chain variable domain comprises a CDR1 that comprises SEQ ID NO: 24; (e) the TCR beta chain variable domain comprises a CDR2 that comprises SEQ ID NO: 25, 26 or 27; and/or (f) the TCR beta chain variable domain comprises a CDR3 that comprises SEQ ID NO: 28. For example, in some cases: (a); (b); (c); (d); (e); (f); (a) and (b); (a) and (c); (a) and (d); (a) and (e); (a) and (f); (b) and (c); (b) and (d); (b) and (e); (b) and (f); (c) and (d); (c) and (e); (c) and (f); (d) and (e); (d) and (f); (e) and (f); (a), (b) and (c); (a), (b) and (d); (a), (b) and (e); (a), (b) and (f); (a), (c) and (d); (a), (c) and (e); (a), (c) and (f); (a), (d) and (e); (a), (d) and (f); (a), (e) and (f); (b), (c) and (d); (b), (c) and (e); (b), (c) and (f); (b), (d) and (e); (b), (d) and (f); (b), (e) and (f); (c), (d) and (e); (c), (d) and (f); (c), (e) and (f); (d), (e) and (f); (a), (b), (c) and (d); (a), (b), (c) and (e); (a), (b), (c) and (f); (a), (b), (d) and (e); (a), (b), (d) and (f); (a), (b), (e) and (f); (a), (c), (d) and (e); (a), (c), (d) and (f); (a), (c), (e) and (f); (a), (d), (e) and (f); (b), (c), (d) and (e); (b), (c), (d) and (f); (b), (c), (e) and (f); (b), (d), (e) and (f); (c), (d), (e) and (f); (a), (b), (c), (d) and (e); (a), (b), (c), (d) and (f); (a), (b), (c), (e) and (f); (a), (b), (d), (e) and (f); (a), (c), (d), (e) and (f); (b), (c), (d), (e) and (f); (a), (b), (c), (d), (e) and (f). For example, in some cases, (a) the TCR alpha chain variable domain comprises a CDR1 that comprises SEQ ID NO: 8, 9, 10, 11, 12, 13 or 14; (b) the TCR alpha chain variable domain comprises a CDR2 that comprises SEQ ID NO: 15; (c) the TCR alpha chain variable domain comprises a CDR3 that comprises SEQ ID NO: 16; (d) the TCR beta chain variable domain comprises a CDR1 that comprises SEQ ID NO: 24; (e) the TCR beta chain variable domain comprises a CDR2 that comprises SEQ ID NO: 25, 26 or 27; and (f) the TCR beta chain variable domain comprises a CDR3 that comprises SEQ ID NO: 28.

The TCR alpha chain variable domain may, for example, comprise a CDR1 that comprises SEQ ID NO: 8, a CDR2 that comprises SEQ ID NO: 15, and a CDR3 that comprises SEQ ID NO: 16. For example, the TCR alpha chain variable domain may comprise SEQ ID NO: 17. The TCR alpha chain variable domain may, for example, comprise a CDR1 that comprises SEQ ID NO: 9, a CDR2 that comprises SEQ ID NO: 15, and a CDR3 that comprises SEQ ID NO: 16. For example, the TCR alpha chain variable domain may comprise SEQ ID NO: 18. The TCR alpha chain variable domain may, for example, comprise a CDR1 that comprises SEQ ID NO: 10, a CDR2 that comprises SEQ ID NO: 15, and a CDR3 that comprises SEQ ID NO: 16. For example, the TCR alpha chain variable domain may comprise SEQ ID NO: 19. The TCR alpha chain variable domain may, for example, comprise a CDR1 that comprises SEQ ID NO: 11, a CDR2 that comprises SEQ ID NO: 15, and a CDR3 that comprises SEQ ID NO: 16. For example, the TCR alpha chain variable domain may comprise SEQ ID NO: 20. The TCR alpha chain variable domain may, for example, comprise a CDR1 that comprises SEQ ID NO: 12, a CDR2 that comprises SEQ ID NO: 15, and a CDR3 that comprises SEQ ID NO: 16. For example, the TCR alpha chain variable domain may comprise SEQ ID NO: 21. The TCR alpha chain variable domain may, for example, comprise a CDR1 that comprises SEQ ID NO: 13, a CDR2 that comprises SEQ ID NO: 15, and a CDR3 that comprises SEQ ID NO: 16. For example, the TCR alpha chain variable domain may comprise SEQ ID NO: 22. The TCR alpha chain variable domain may, for example, comprise a CDR1 that comprises SEQ ID NO: 14, a CDR2 that comprises SEQ ID NO: 15, and a CDR3 that comprises SEQ ID NO: 16. For example, the TCR alpha chain variable domain may comprise SEQ ID NO: 23.

Any alpha chain variable domain disclosed herein may comprise a signal peptide at its N-terminus. An alpha chain variable domain may, for example, be expressed with an N-terminal signal peptide that is cleaved prior to expression at the surface of the T cell. The signal peptide may, for example, comprise or consist of SEQ ID NO: 38. The alpha chain variable domain may, for instance comprise SEQ ID NO: 38 and SEQ ID NO: 17, wherein the C-terminal “S” of SEQ ID NO: 38 is directly joined to the N-terminal “G” of SEQ ID NO: 17 by a peptide bond. The alpha chain variable domain may, for instance comprise SEQ ID NO: 38 and SEQ ID NO: 18, wherein the C-terminal “S” of SEQ ID NO: 38 is directly joined to the N-terminal “G” of SEQ ID NO: 18 by a peptide bond. The alpha chain variable domain may, for instance comprise SEQ ID NO: 38 and SEQ ID NO: 19, wherein the C-terminal “S” of SEQ ID NO: 38 is directly joined to the N-terminal “G” of SEQ ID NO: 19 by a peptide bond. The alpha chain variable domain may, for instance comprise SEQ ID NO: 38 and SEQ ID NO: 20, wherein the C-terminal “S” of SEQ ID NO: 38 is directly joined to the N-terminal “G” of SEQ ID NO: 20 by a peptide bond. The alpha chain variable domain may, for instance comprise SEQ ID NO: 38 and SEQ ID NO: 21, wherein the C-terminal “S” of SEQ ID NO: 38 is directly joined to the N-terminal “G” of SEQ ID NO: 21 by a peptide bond. The alpha chain variable domain may, for instance comprise SEQ ID NO: 38 and SEQ ID NO: 22, wherein the C-terminal “S” of SEQ ID NO: 38 is directly joined to the N-terminal “G” of SEQ ID NO: 22 by a peptide bond. The alpha chain variable domain may, for instance comprise SEQ ID NO: 38 and SEQ ID NO: 23, wherein the C-terminal “S” of SEQ ID NO: 38 is directly joined to the N-terminal “G” of SEQ ID NO: 23 by a peptide bond.

The TCR beta chain variable domain may, for example, comprise a CDR1 that comprises SEQ ID NO: 24, a CDR2 that comprises SEQ ID NO: 25, and a CDR3 that comprises SEQ ID NO: 28. For example, the TCR beta chain variable domain may comprise SEQ ID NO: 29. The TCR beta chain variable domain may, for example, comprise a CDR1 that comprises SEQ ID NO: 24, a CDR2 that comprises SEQ ID NO: 26, and a CDR3 that comprises SEQ ID NO: 28. For example, the TCR beta chain variable domain may comprise SEQ ID NO: 30. The TCR beta chain variable domain may, for example, comprise a CDR1 that comprises SEQ ID NO: 24, a CDR2 that comprises SEQ ID NO: 27, and a CDR3 that comprises SEQ ID NO: 28. For example, the TCR beta chain variable domain may comprise SEQ ID NO: 31.

Any beta chain variable domain disclosed herein may comprise a signal peptide at its N-terminus. A beta chain variable domain may, for example, be expressed with an N-terminal signal peptide that is cleaved prior to expression at the surface of the T cell. The signal peptide may, for example, comprise or consist of SEQ ID NO: 39. The beta chain variable domain may, for instance comprise SEQ ID NO: 39 and SEQ ID NO: 29, wherein the C-terminal “L” of SEQ ID NO: 39 is directly joined to the N-terminal “E” of SEQ ID NO: 29 by a peptide bond. The beta chain variable domain may, for instance comprise SEQ ID NO: 39 and SEQ ID NO: 30, wherein the C-terminal “L” of SEQ ID NO: 39 is directly joined to the N-terminal “E” of SEQ ID NO: 30 by a peptide bond. The beta chain variable domain may, for instance comprise SEQ ID NO: 39 and SEQ ID NO: 31, wherein the C-terminal “L” of SEQ ID NO: 39 is directly joined to the N-terminal “E” of SEQ ID NO: 31 by a peptide bond.

The TCR may, for example comprise a TCR alpha chain variable domain comprising a CDR1 that comprises SEQ ID NO: 8, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 25, and a CDR3 that comprises SEQ ID NO: 28. For example, the TCR may comprise a TCR alpha chain variable domain that comprises SEQ ID NO: 17, and a TCR beta chain variable domain that comprises SEQ ID NO: 29. The TCR may, for example, comprise a TCR alpha chain variable domain comprising a CDR1 that comprises SEQ ID NO: 8, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 26, and a CDR3 that comprises SEQ ID NO: 28. For example, the TCR may comprise a TCR alpha chain variable domain that comprises SEQ ID NO: 17, and a TCR beta chain variable domain that comprises SEQ ID NO: 30. The TCR may, for example, comprise a TCR alpha chain variable domain comprising a CDR1 that comprises SEQ ID NO: 8, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 17, and a TCR beta chain variable domain that comprises SEQ ID NO: 31. The TCR may, for example, comprise a TCR alpha chain variable domain comprising a CDR1 that comprises SEQ ID NO: 9, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 18, and a TCR beta chain variable domain that comprises SEQ ID NO: 31. The TCR may, for example, comprise a TCR alpha chain variable domain comprising a CDR1 that comprises SEQ ID NO: 10, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 19, and a TCR beta chain variable domain that comprises SEQ ID NO: 31. The TCR may, for example, comprise a TCR alpha chain variable domain comprising a CDR1 that comprises SEQ ID NO: 11, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 20, and a TCR beta chain variable domain that comprises SEQ ID NO: 31. The TCR may, for example, comprise a TCR alpha chain variable domain comprising a CDR1 that comprises SEQ ID NO: 12, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 21, and a TCR beta chain variable domain that comprises SEQ ID NO: 31. The TCR may, for example, comprise a TCR alpha chain variable domain comprising a CDR1 that comprises SEQ ID NO: 13, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 22, and a TCR beta chain variable domain that comprises SEQ ID NO: 31. The TCR may, for example, comprise a TCR alpha chain variable domain comprising a CDR1 that comprises SEQ ID NO: 14, a CDR2 that comprises SEQ ID NO: 15, and a CDR3 that comprises SEQ ID NO: 16, and a TCR beta chain variable domain comprising a CDR1 that comprises SEQ ID NO: 24, a CDR2 that comprises SEQ ID NO: 27, and a CDR3 that comprises SEQ ID NO: 28. For example, the TCR may comprise a TCR alpha chain variable domain that comprises SEQ ID NO: 23, and a TCR beta chain variable domain that comprises SEQ ID NO: 31. In any case, a signal peptide may be present at the N-terminus of the alpha chain variable domain and/or the N-terminus of the beta chain variable domain. The alpha chain signal peptide may comprise SEQ ID NO: 38. The beta chain signal peptide may comprise SEQ ID NO: 39.

Any of the TCR alpha chain variable domains and TCR beta chain variable domains defined above in terms of their CDR sequences may further comprise one or more framework sequences. The one or more framework sequences may, for example, be found (1) before CDR1, (2) between CDR1 and CDR2, (3) between CDR2 and CDR3, and/or (4) after CDR3. The one or more framework sequences may, for example, be found: (1); (2); (3); (4); (1) and (2); (1) and (3); (1) and (4); (2) and (3); (2) and (4), (3) and (4); (1), (2) and (3); (1), (2) and (4); (1), (3) and (4); (2), (3) and (4); or (1), (2), (3) and (4). In the TCR alpha chain variable domain, the one or more framework sequences may, for example, have at least 60%, at least 70%, at least 75%, at least 80%, at least 90%, at least 95%, at least 97%, at least 98% or at least 99% sequence identity to one or more corresponding framework sequences in SEQ ID NO: 17. In the TCR beta chain variable domain, the one or more framework sequences may have at least 60%, at least 70%, at least 75%, at least 80%, at least 90%, at least 95%, at least 97%, at least 98% or at least 99% sequence identity to one or more corresponding framework sequences in SEQ ID NO: 29.

### Constant Domain

The TCR may, for example, comprise a TCR alpha chain constant domain. The TCR may, for example, comprise a TCR beta chain constant domain. The TCR may, for example, comprise a TCR alpha chain constant domain and a TCR beta chain constant domain.

The TCR may comprise any TCR alpha chain constant domain. The TCR alpha chain constant domain may, for example, comprise a TRAC constant domain sequence. The TRAC constant domain sequence may, for example, comprise SEQ ID NO: 2 or an amino acid sequence that has at least 60%, at least 70%, at least 75%, at least 80%, at least 90%, at least 95%, at least 97%, at least 98%, or at least 99% sequence identity to SEQ ID NO: 2. SEQ ID NO: 2 represents the native form of TRAC. The TRAC constant domain sequence may, for example, comprise SEQ ID NO: 32 or an amino acid sequence that has at least 60%, at least 70%, at least 75%, at least 80%, at least 90%, at least 95%, at least 97%, at least 98%, or at least 99% sequence identity to SEQ ID NO: 32. SEQ ID NO: 32 is a version of SEQ ID NO: 2 without the N-terminal “N”.

In another aspect of the disclosure, the TRAC constant domain sequence may, for example, comprise a variant of SEQ ID NO: 2 in which the threonine at position 48 is substituted for a cysteine. That is, the TRAC constant domain sequence may comprise a T48C mutant of SEQ ID NO: 2. For instance, the TRAC constant domain sequence may comprise SEQ ID NO: 3 or an amino acid sequence that has at least 60%, at least 70%, at least 75%, at least 80%, at least 90%, at least 95%, at least 97%, at least 98%, or at least 99% sequence identity to SEQ ID NO: 3. The TRAC constant domain sequence may comprise SEQ ID NO: 33 or an amino acid sequence that has at least 60%, at least 70%, at least 75%, at least 80%, at least 90%, at least 95%, at least 97%, at least 98%, or at least 99% sequence identity to SEQ ID NO: 33. SEQ ID NO: 33 is a version of SEQ ID NO: 3 without the N-terminal “N”.

In a further aspect of the disclosure, the TRAC constant domain sequence may encode a soluble version of TRAC. For instance, the TRAC constant domain sequence may comprise SEQ ID NO: 4 or an amino acid sequence that has at least 60%, at least 70%, at least 75%, at least 80%, at least 90%, at least 95%, at least 97%, at least 98%, or at least 99% sequence identity to SEQ ID NO: 4. The TRAC constant domain sequence may comprise SEQ ID NO: 34 or an amino acid sequence that has at least 60%, at least 70%, at least 75%, at least 80%, at least 90%, at least 95%, at least 97%, at least 98%, or at least 99% sequence identity to SEQ ID NO: 34. SEQ ID NO: 34 is a version of SEQ ID NO: 4 without the N-terminal “N”.

In one aspect of the disclosure, the TRAC constant domain sequence may, for example, be modified by truncation or substitution to delete the native disulfide bond between Cys4 of exon 2 of TRAC and Cys2 of exon 2 of TRBC1 or TRBC2.

The various TCR alpha chain constant domains described above may be used interchangeably. That is, any TCR alpha chain constant domain may combined with any TCR alpha chain variable domain to form a TCR alpha chain.

The TCR may comprise any TCR beta chain constant domain. The TCR beta chain constant domain may, for example, comprise TRBC2 constant domain sequence. The TRBC2 constant domain sequence may, for example, comprise SEQ ID NO: 5 or an amino acid sequence that has at least 60%, at least 70%, at least 75%, at least 80%, at least 90%, at least 95%, at least 97%, at least 98%, or at least 99% sequence identity to SEQ ID NO: 5. SEQ ID NO: 5 represents the native form of TRBC2. The TRBC2 constant domain sequence may, for example, comprise SEQ ID NO: 35 or an amino acid sequence that has at least 60%, at least 70%, at least 75%, at least 80%, at least 90%, at least 95%, at least 97%, at least 98%, or at least 99% sequence identity to SEQ ID NO: 35. SEQ ID NO: 35 is a version of SEQ ID NO: 5 without the N-terminal “E”.

In another aspect of the disclosure, the TRAC constant domain sequence may, for example, comprise a variant of SEQ ID NO: 5 in which the serine at position 57 is substituted for a cysteine. That is, the TRBC2 constant domain sequence may comprise a S57C mutant of SEQ ID NO 5. For instance, the TRBC2 constant domain sequence may comprise SEQ ID NO: 6 or an amino acid sequence that has at least 60%, at least 70%, at least 75%, at least 80%, at least 90%, at least 95%, at least 97%, at least 98%, or at least 99% sequence identity to SEQ ID NO: 6. The TRBC2 constant domain sequence may comprise SEQ ID NO: 36 or an amino acid sequence that has at least 60%, at least 70%, at least 75%, at least 80%, at least 90%, at least 95%, at least 97%, at least 98%, or at least 99% sequence identity to SEQ ID NO: 36. SEQ ID NO: 36 is a version of SEQ ID NO: 6 without the N-terminal “E”.

In a further aspect of the disclosure, the TCR beta chain constant domain may be a soluble TCR beta chain constant domain. For instance, the TCR beta chain constant domain may comprise a TRBC1/2 splice, such as SEQ ID NO: 7 or an amino acid sequence that has at least 60%, at least 70%, at least 75%, at least 80%, at least 90%, at least 95%, at least 97%, at least 98%, or at least 99% sequence identity to SEQ ID NO: 7. The TCR beta chain constant domain may comprise a TRBC1/2 splice, such as SEQ ID NO: 37 or an amino acid sequence that has at least 60%, at least 70%, at least 75%, at least 80%, at least 90%, at least 95%, at least 97%, at least 98%, or at least 99% sequence identity to SEQ ID NO: 37. SEQ ID NO: 37 is a version of SEQ ID NO: 7 without the N-terminal “E”.

In one aspect of the disclosure, the TCR beta chain constant domain (e.g. TCRB2 or TCRB1/2) sequence may be modified by truncation or substitution to delete the native disulfide bond between Cys4 of exon 2 of TRAC and Cys2 of exon 2 of TRBC1 or TRBC2.

In one aspect of the disclosure, the TCR comprises a TCR alpha chain constant domain with a T48C substitution and a TCR beta chain constant domain with a S57C substitution. The T48C substitution in the TCR alpha chain and the S57C substitution in the beta chain are advantageous as these are the cysteines which form a disulfide bond between the alpha and beta constant domains of the TCR. The addition of these cysteines allows for the formation of a disulfide bond between the alpha and beta constant domains of the TCR and enhance preferential pairing of the alpha and beta chains (Blood. 2007 Mar. 15; 109(6): 2331-2338).

The TCR according to the present disclosure may, for example, be an alpha-beta heterodimer. An alpha-beta heterodimer may be defined as a dimer that comprises a TCR alpha chain and a TCR beta chain. Typically, a TCR alpha chain comprises a TCR alpha chain variable domain and a TCR alpha chain constant domain. Typically, a TCR beta chain comprises a TCR beta chain variable domain and a TCR beta chain constant domain. Therefore, the TCR of the disclosure may be an alpha-beta heterodimer that comprises (a) a TCR alpha chain comprising (i) a TCR alpha chain variable domain defined herein and (ii) a TCR alpha chain constant domain; and (b) a TCR beta chain comprising (i) a TCR beta chain variable domain defined herein and (ii) a TCR beta chain constant domain.

In another aspect of the disclosure, the TCR may be a single chain TCR of the structure Vα-L-Vβ, Vβ-L-Vα, Vα-Cα-L-Vβ, or Vα-L-Vβ-Cβ; wherein Vα is the TCR alpha chain variable domain, Vβ is the TCR beta chain variable domain, Cα is TCR alpha chain constant domain, Cβ is a TCR beta chain constant domain, and L is a linker sequence.

In any case, the TCR of the disclosure may be in a format suitable for expression on the surface of a T cell. Alternatively the TCR of the disclosure may be soluble. Use of a stable disulfide linked soluble TCR as a reference TCR enables more convenient assessment of binding affinity and binding half-life. For example, for the purpose of providing a measure of binding, binding affinity, specificity, selectivity it is convenient to use a soluble TCR. As outlined elsewhere, soluble TCRs can also be used for therapeutic purposes, such as targeting a cytotoxic agent, which is coupled to the soluble TCR, to a tumour site.

The soluble nature of a TCR may be conferred by the presence of a soluble TCR alpha chain constant domain and/or a soluble TCR beta chain constant domain. Thus, a soluble TCR may comprise a soluble TCR alpha chain constant domain. Soluble TCR alpha chain constant domains are known in the art and described above. Soluble TCRs may, for example, comprise an alpha chain constant domain comprising a sequence which has at least 60%, at least 70%, at least 75%, at least 80%, at least 90%, at least 95%, at least 97%, at least 98% or at least 99% sequence identity to SEQ ID NO: 4. A soluble TCR may comprise a soluble TCR beta chain constant domain. Soluble TCR beta chain constant domains are known in the art and described above. Soluble TCRs may, for example, comprise a beta chain constant domain comprising a sequence which has at least 60%, at least 70%, at least 75%, at least 80%, at least 90%, at least 95%, at least 97%, at least 98% or at least 99% sequence identity to SEQ ID NO: 7. A soluble TCR may comprise a soluble TCR alpha chain constant domain and a soluble TCR beta chain constant domain. Soluble TCRs may comprise an alpha chain constant domain comprising the sequence of SEQ ID NO: 4 and a beta chain constant domain comprising the sequence of SEQ ID NO: 7.

### Complexes

The disclosure also provides a complex comprising (a) a TCR of the disclosure, and (b) a detectable label, a therapeutic agent or a pharmacokinetic modifying moiety.

The detectable label may, for example, comprise a fluorescent label, radiolabel, enzyme, nucleic acid probe or contrast reagent.

The therapeutic agent may, for example, comprise an immunomodulator, a radioactive compound, an enzyme (perforin for example) or a chemotherapeutic agent. Other suitable therapeutic agents include:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin;
peptide cytotoxins, i.e. proteins or fragments thereof with the ability to kill mammalian cells. For example, ricin, diphtheria toxin, pseudomonas bacterial exotoxin A, DNase and RNase;
radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
immuno-stimulants, i.e. immune effector molecules which stimulate immune response. For example, cytokines such as IL-2 and IFN-γ,
Superantigens and mutants thereof;
TCR-HLA fusions;
chemokines such as IL-8, platelet factor 4, melanoma growth stimulatory protein, etc;
antibodies or fragments thereof, including anti-T cell or NK cell determinant antibodies (e.g. anti-CD3, anti-CD28 or anti-CD16);
alternative protein scaffolds with antibody like binding characteristics
complement activators;
xenogeneic protein domains, allogeneic protein domains, viral/bacterial protein domains, viral/bacterial peptides.

A pharmacokinetic modifying moiety may, for example, confer PEGylation. The pharmacokinetic modifying moiety may, for example, comprise PEG.

In another aspect, the TCR of the disclosure may be included in a complex comprising several TCRs, to form a multivalent TCR complex. There are a number of human proteins that contain a multimerisation domain that may be used in the production of multivalent TCR complexes. For example the tetramerisation domain of p53 which has been utilised to produce tetramers of scFv antibody fragments which exhibit increased serum persistence and significantly reduce off-rate compared to the monomeric scFv fragment. (Willuda et al. (2001) J. Biol. Chem. 276 (17) 14385-14392). Haemoglobin also has a tetramerisation domain that could potentially be used for this kind of application. A multivalent TCR complex of the disclosure may have enhanced binding capability for the SLLQHLIGL (SEQ ID NO: 1) HLA-A*02 complex compared to a non-multimeric TCRs of the disclosure. Thus, the TCRs may form part of a multivalent TCR complex. Such multivalent TCR complexes are particularly useful for tracking or targeting cells presenting particular antigens in vitro or in vivo, and are also useful as intermediates for the production of further multivalent TCR complexes having such uses.

### Post Translational Modifications

TCRs of the disclosure may be subject to post translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes.

Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody-based therapeutics. (Jefferis R., Nat Rev Drug Discov. 2009 March; 8(3):226-34).

For soluble TCRs of the disclosure glycosylation may be controlled in vivo, by using particular cell lines for example, or in vitro, by chemical modification. Such modifications are desirable, since glycosylation can improve pharmacokinetics, reduce immunogenicity and more closely mimic a native human protein (Sinclair AM and Elliott S., Pharm Sci. 2005 August; 94(8): 1626-35).

### Binding Properties

The TCRs of the disclosure are capable of binding a complex comprising the PRAME peptide sequence SLLQHLIGL (SEQ ID NO: 1) and HLA-A*02. The binding may be specific. Specificity refers to the strength of binding between the TCR and its target antigen. Specificity may be described by a dissociation constant, Kd, the ratio between bound and unbound states for the receptor-ligand system. Typically, the fewer different antigens the TCR is capable of binding other than SEQ ID NO: 1, the greater its binding specificity.

The TCR may, for example, bind to the complex comprising SEQ ID NO: 1 and HLA-A*02 with a dissociation constant (Kd) of between 0.01 μM and 100 μM, between 0.01 μM and 50 μM, between 0.01 μM and 20 μM, between 10 μM and 1000 μM, between 10 μM and 500 μM, or between 50 μM and 500 μM. For instance, in a preferred aspect of the disclosure, the TCR binds to the complex comprising SEQ ID NO: 1 and HLA-A*02 with a Kd of between 0.05 μM to 20.0 μM. The Kd may, for example, be measured using surface plasmon resonance, optionally at 25° C., optionally between a pH of 6.5 and 6.9 or 7.0 and 7.5. The dissociation constant, Kd or koff/kon may be determined by experimentally measuring the dissociation rate constant, koff, and the association rate constant, kon. A TCR dissociation constant may be measured using a soluble form of the TCR, wherein the TCR comprises a TCR alpha chain variable domain and a TCR beta chain variable domain.

The efficacy of TCRs can be quantified by measuring the activation of T cells expressing the TCRs when contacted with antigen-presenting cells presenting the target antigen (in the present disclosure, a complex comprising SEQ ID NO: 1 and HLA-A*02). The T cells expressing a TCR disclosed herein may specifically bind to HLA-A*02-expressing APCs pulsed with a peptide comprising the sequence of SEQ ID NO: 1. In some aspects of the disclosure, TCR efficacy may be quantified by co-culturing the T cells and the APCs at a 1:1 effector-to-target cell ratio. In some aspects of the disclosure, half maximal effective concentration (EC50) can be determined by measuring the presence of CD69 on the T cells. CD69 is an activation marker for T cells (Simms et al. Clinical and Diagnostic Laboratory Immunology, May 1996, p. 301-304; Maino et a. Cytometry 1995, 20:127-133; Abraham R T, Weiss A. Nat Rev Immunol., 2004, pp. 301-8). In some aspects of the disclosure, the EC50 is determined using a CD4+ T cell population. An exemplary CD69 T-cell activation assay is described in Example 1. The EC50 of the TCR may, for example, be less that be less than 1 mM, less than 100 μM, less than 50 μM, less than 40 μM, less than 30 μM, less than 20 μM, less than 10 μM, less than 5 μM, less than 1 μM less than 100 nM, less than 50 nM, less than 40 nM, less than 30 nM, less than 20 nM, less than 10 nM, less than 5 nM, less than 1 nM, less than 100 pM, less than 50 pM, less than 40 pM, less than 30 pM, less than 20 pM, less than 10 pM, less than 5 pM, or less than 1 pM. In some cases, the EC50 of the TCR is less than 20 nM. In some cases, the EC50 of the TCR is between 1 pM and 1 mM, such as from 5 pM to 100 μM, from 10 pM to 50 μM, from 20 pM to 40 μM, from 30 pM to 30 μM, from 40 pM to 20 μM, from 50 pM to 10 μM, from 100 pM to 5 μM, from 1 nM to 1 μM, from 5 nM to 100 nM, from 10 nM to 50 nM, or from 20 nM to 40 nM. In some cases, the EC50 of the TCR is between 50 pM and 20 nM.

### Nucleic Acids

The disclosure provides a nucleic acid encoding any TCR of the disclosure. The nucleic acid may encode the TCR in a single open reading frame. That is, the nucleic acid may encode the alpha chain variable region and the beta chain variable region in a single ORF. The nucleic acid may further encode (a) an alpha chain constant domain and/or (b) a beta chain constant domain in the single ORF. In other words, the nucleic acid may encode the TCR alpha chain and the TCR beta chain in the single ORF.

Alternatively, the nucleic acid may comprise (i) a first open reading frame encoding a TCR alpha chain comprising the TCR alpha chain variable domain and optionally a TCR alpha chain constant domain, and (ii) a second open reading frame encoding a TCR beta chain comprising the TCR beta chain variable domain and optionally a TCR beta chain constant domain. That is, the nucleic acid may encode the alpha chain variable region (and optionally an alpha chain constant domain) and the beta chain variable region (and optionally a beta chain constant domain) in separate ORFs. In other words, the nucleic acid may encode the TCR alpha chain and the TCR beta chain in separate ORFs.

The disclosure also provides a nucleic acid encoding a TCR alpha chain variable domain of any TCR of the disclosure. The nucleic acid may further encode a TCR alpha chain constant domain. The TCR alpha chain constant domain may be any TCR alpha chain constant domain, such as a TCR alpha chain constant domain described herein.

The disclosure further provides a nucleic acid encoding a TCR beta chain variable domain of any TCR of the disclosure. The nucleic acid may further encode a TCR beta chain constant domain. The TCR beta chain constant domain may be any TCR beta chain constant domain, such as a TCR beta chain constant domain described herein.

In any case, the nucleic acid may be isolated. The nucleic acid may be non-naturally occurring. The nucleic acid may be non-naturally occurring purified. The nucleic acid may be non-naturally occurring engineered. The nucleic acid may be non-naturally occurring recombinant.

The nucleic acid can be DNA, such as cDNA. The nucleic acid may be RNA.

### Vectors

Also provided are vectors which may comprise a nucleic acid of the disclosure.

The vector may be an expression vector, which may further comprise regulatory elements for the expression of the product encoded by the nucleic acid of the disclosure.

### Viral Particles

The disclosure further provides a viral particle comprising a nucleic acid or vector of the disclosure. The viral particle may be suitable for transducing cells, such as T cells, with a sequence encoding a TCR, TCR alpha chain, TCR beta chain, TCR alpha chain variable region, or TCR beta chain variable region described herein.

The viral particle may, for example, be a retroviral particle or a lentiviral particle.

Viral particles for transduction may be produced according to known methods. For example, HEK293T cells may be transfected with plasmids encoding viral packaging and envelope elements as well as a lentiviral vector comprising a nucleic acid of the disclosure. A VSVg-pseudotyped viral vector comprising the nucleic acid of the disclosure may be produced in combination with the viral envelope glycoprotein G of the Vesicular stomatitis virus (VSVg) to produce a pseudotyped virus particle.

### Cells

The disclosure provides a cell that expresses a TCR of the disclosure.

The disclosure also provides a cell that comprises at least one nucleic acid of the disclosure. The cell may comprise (i) a first nucleic acid of the disclosure that encodes a TCR alpha chain variable domain of a TCR of the disclosure and optionally a TCR alpha chain constant domain, and (ii) a second nucleic acid of the disclosure that encodes a TCR beta chain variable domain of a TCR of the disclosure and optionally a TCR beta chain constant domain. Thus, the cell may be a cell that is transduced with one or more nucleic acids that together encode a TCR of the disclosure.

The disclosure further provides a cell that comprises at least one vector of the disclosure. The cell may comprise (i) a first vector that comprises a nucleic acid of the disclosure that encodes a TCR alpha chain variable domain of a TCR of the disclosure and optionally a TCR alpha chain constant domain, and (ii) a second vector that comprises a nucleic acid of the disclosure that encodes a TCR beta chain variable domain of a TCR of the disclosure and optionally a TCR beta chain constant domain. Thus, the cell may be a cell that is transduced with one or more vectors that together encode a TCR of the disclosure.

In any case, the cell may be an isolated cell. The cell may be a non-naturally occurring cell. In other words, the cell may be an engineered cell.

The cell may be a T cell. The T cell may be a CD4+ T cell. That is, the T cell may be a T cell that expresses an endogenous CD4 co-receptor. The T cell may be a CD8+ T cell. That is, the T cell may be a T cell that expresses an endogenous CD8 co-receptor.

In any case, the T cell may express a heterologous CD8 co-receptor. In the context of the present disclosure, the term “heterologous” refers to a polypeptide or nucleic acid that is foreign to a particular biological system (such as a T cell), i.e. that is not naturally present in that system. A “heterologous” polypeptide or nucleic acid may be introduced to the system by artificial or recombinant means. Accordingly, heterologous expression of a TCR may alter the specificity of a T cell. Heterologous expression of a CD8 co-receptor may endow the T cell with functions associated with the CD8 co-receptor. For instance, expression of a heterologous CD8 co-receptor may confer upon the T cell an improved affinity and/or avidity for PRAME, and/or improved activation upon binding to PRAME. Methods for determining affinity, avidity and T cell activation are well-known in the art. Expression of a heterologous CD8 co-receptor may confer upon the T cell an improved or increased expression of CD40L, cytokine production, cytotoxic activity, induction of dendritic cell maturation or induction of dendritic cell cytokine production, for instance in response to antigen (PRAME) binding. Improvements or increases may be relative to modified T cells that comprise a heterologous TCR that binds to PRAME but which lack a heterologous CD8 co-receptor.

The cells of the disclosure have utility in adoptive therapy. For instance, cells of the disclosure are suitable for use in adoptive therapy-based treatment of cancer.

The cell may be allogeneic. For example, the cell may be allogeneic with respect to an individual into which it is to be administered. The cell may be autologous. For example, the cell may be autologous with respect to an individual into which it is to be administered. Autologous cells may be produced by modifying endogenous cells, such as peripheral blood mononuclear cells (PBMCs), obtained from the individual. Methods for modification are known in the art.

The cell of the disclosure may further comprise one or more exogenous or recombinant co-stimulatory ligands. The cell may, for example, be transduced with one or more exogenous or recombinant co-stimulatory ligands.

The cell may comprise two or more, three or more, or four or more exogenous or recombinant co-stimulatory ligands.

The cell may express the one or more exogenous or recombinant co-stimulatory ligands. The cell may co-express (a) the TCR of the disclosure, the product encoded by the nucleic acid(s) of the disclosure, or the product encoded by the vector(s) of the disclosure, and (b) the one or more exogenous or recombinant co-stimulatory ligands. The combination of (a) and (b) may facilitate provision of a non-antigen-specific signal and antigen-specific activation of the cell.

Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, but are not limited to, nerve growth factor (NGF), CD40L (CD40L)/CD154, CD137L/4-1BBL, TNF-alpha, CD134L/OX40L/CD252, CD27L/CD70, Fas ligand (FasL),CD30L/CD153, tumour necrosis factor beta (TNFb)/lymphotoxin-alpha (LTa),lymphotoxin-beta (TTb), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof.

In one aspect, the exogenous or recombinant co-stimulatory ligands may comprise 4-1BBL or CD80. In one aspect, the exogenous or recombinant co-stimulatory ligand may be 4-1BBL. In one aspect, the exogenous or recombinant co-stimulatory ligands may be 4-1BBL and CD80.

In one aspect the one or more exogenous or recombinant co-stimulatory ligands comprise a cyclic-AMP phosphodiesterase. In one aspect the one or more exogenous or recombinant co-stimulatory ligands comprise a transforming growth factor beta (TGFbeta) receptor, such as dominant-negative TGFbeta receptor II.

The cell may be engineered to release cytokines which have a positive effect on the cytolytic activity of the cell. Such cytokines include, but are not limited to interleukin-7, interleukin-15 and interleukin-21.

### Pharmaceutical Compositions

Also provided is a pharmaceutical composition comprising the TCR of the disclosure, the nucleic acid of the disclosure, or the cell of the disclosure. The pharmaceutical composition further comprises one or more pharmaceutically acceptable carrier or excipient. Suitable carriers and excipients include, for example, water, saline, dextrose, glycerol, and the like and combinations thereof. In addition, if desired, the pharmaceutical compositions may contain minor amounts of auxiliary substances such as wetting or emulsifying agents, and/or pH buffering agents.

The pharmaceutical composition may be provided in unit dosage form. The pharmaceutical composition may be provided in a sealed container and may be provided as part of a kit. Such a kit may include instructions for use. It may include a plurality of said unit dosage forms.

### Treatment of Cancer

The disclosure provides the TCR, nucleic acid, cell or pharmaceutical composition of the disclosure, for use in a method of treating a disease in an individual, the method comprising administering the TCR, nucleic acid, cell or pharmaceutical composition to the individual. The disclosure also provides a method of treating a disease in an individual, comprising administering to the individual the TCR, nucleic acid, cell or pharmaceutical composition of the disclosure.

As PRAME is a cancer antigen, the disease may for example be cancer. For instance, the disease may be a solid tumour. The disease may, for example, be selected from selected from melanoma, leukaemia, lymphoma, lung cancer, breast cancer, ovarian cancer, endometrial cancer, oesophageal cancer, bladder cancer and head and neck cancers. The lung cancer may, for example, be non-small cell lung cancer or small cell lung cancer. The breast cancer may, for example, be triple negative breast cancer.

The cancer may be a PRAME-expressing cancer. “PRAME expressing cancer” refers to any cancer in which cancer cells exhibit expression of the PRAME antigen. Such cancers include any of these listed in the preceding paragraph.

Preferably, the individual is a human. Non-human mammals, especially mammals that are conventionally used as models for demonstrating therapeutic efficacy in humans (e.g. murine, primate, porcine, canine, or rabbit animals) may though be treatable according to the method described above.

Treatment may aim to achieve a therapeutic effect. The therapeutic effect may, for instance, comprise, the inhibition or delay of the progress of the disease. For instance, the therapeutic effect may include a reduction in the rate of progress; a halt in the rate of progress; amelioration of the disease; cure or remission (whether partial or total) of the disease; preventing, delaying, abating or arresting one or more symptoms and/or signs of the disease, and/or prolonging survival of a subject or patient beyond that expected in the absence of treatment or for example in comparison to a control or control individual. Therapeutic effect may, for example, be measured as TTP (time to progression), OS (Overall Survival), or PFS (Progression Free Survival).

The therapeutic effect may, for instance, be an anti-cancer effect. An anti-cancer effect may include a reduction in the rate of tumour growth, a decrease in tumour volume, a decrease in the number of tumour cells, a decrease in the number of metastases, an increase in life expectancy, or amelioration of various physiological symptoms associated with the cancerous condition. This may be measured in vivo or in vitro optionally in comparison to a control or control individual comprising treatment with vehicle or no treatment.

Treatment may aim to achieve a prophylactic effect. For example, an individual susceptible to or at risk of the occurrence or re-occurrence of disease (e.g. cancer) may be treated as described herein. Such treatment may prevent or delay the occurrence or re-occurrence of disease in the individual, optionally for example in comparison to a control or control individual.

Administration may be by any convenient route. Administration may, for example, be parenteral, for example, by infusion; intravenous; or subcutaneous. Infusion may involve the administration of the therapeutic through a needle or catheter.

Typically, cells such as T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

The therapeutic is administered in a manner compatible with its dosage formulation and in such amount will be therapeutically effective. The quantity to be administered depends on the subject to be treated, the disease to be treated, and the capacity of the individual's immune system. Precise amounts of therapeutic required to be administered may depend on the judgement of the practitioner and may be peculiar to each subject. Determining the optimal dosage will generally involve the balancing of the level of therapeutic benefit against any risk or deleterious side effects of the treatments. The selected dosage level will depend on a variety of factors including, but not limited to, the activity of the particular therapeutic, the route of administration, the time of administration, the rate of loss or inactivation of the therapeutic, the duration of the treatment, other drugs, compounds, and/or materials used in combination, and the age, sex, weight, condition, general health, and prior medical history of the patient.

When the therapeutic comprises T cells, the number of T cells administered may, for example, be from about 10⁵ to about 10¹⁰ per Kg body weight, for instance 2×10⁵ to 2×10¹⁰ cells per individual. Administration may, for example, be over the course of around 30 minutes, with treatment repeated as necessary. The treatment may be repeated at intervals of days to weeks. The appropriate dosages of T cells, and compositions comprising T cells, can vary from patient to patient.

The method may comprise administering a further therapeutic agent to the individual. The identity of the further therapeutic agent will depend on the disease to be treated. Any known therapeutic agent for the disease may be used in the method. When the disease is cancer, the further therapeutic agent may, for example, be a chemotherapeutic agent or an immunomodulatory agent. The immunomodulatory agent may, for example, comprise an antibody, an immune cell, or a checkpoint inhibitor.

### Method of Making Cells

Also provided is a method of making a cell of the disclosure. The method comprises introducing to a cell at least one nucleic acid of the disclosure or at least one vector of the disclosure. The cell may be any suitable cell type. The cell may be a cell as described above, for example. Preferably, the cell is a T cell. The at least one nucleic acid or at least one vector may, for example, be comprised in a virus particle. Virus particles are described above.

The method may further comprise culturing the cell such that the cell proliferates to form a population. Suitable culture conditions are well-known in the art for various cell types, such as T cells. The method may also comprise isolating or purifying cells of the disclosure from the produced population. Methods for isolating and purifying desired cells from a population are well known in the art and include, for example, magnetic cell sorting and fluorescence activated cell sorting.

### EXAMPLES

### Example 1

### Jurkat CD69 T-Cell Activation Assay

The Jurkat cell line and its derivatives were used extensively during the late 20^{th} century to delineate the major components of the T cell signalling pathway (1). CD69 is a C-type lectin commonly used as an early activation marker in both Jurkat and primary T cells—it is rapidly upregulated following TCR signalling and can be readily detected using flow cytometry. Transgenic TCRs may be expressed in Jurkat cells using lentiviral particles as a vector for transduction of the TCR transgene. Suitable antigen-presenting cells for activation of TCR-transduced Jurkat cells include cell lines expressing the cognate HLA allele and antigen of interest, and the HLA-A*02⁺, TAP-deficient LCL-B/T-cell hybrid line ‘T2’ pulsed with exogenous target.

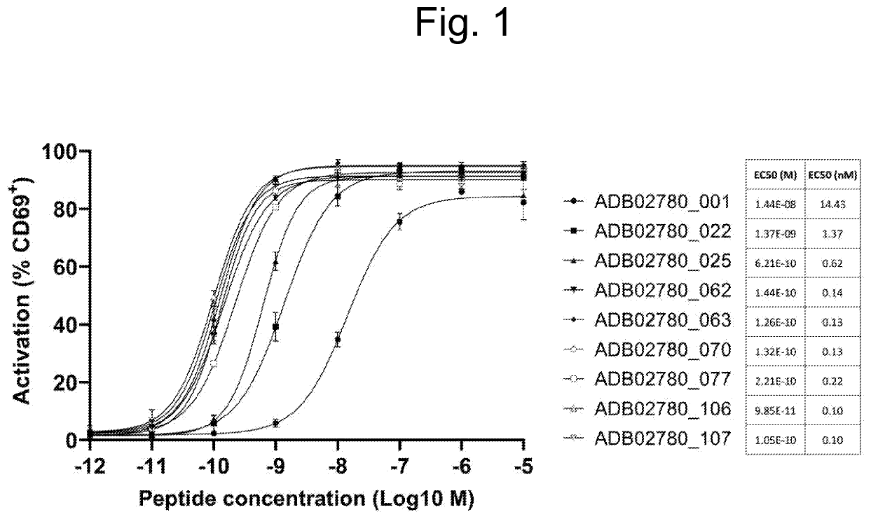

TCR-transduced Jurkat cells were co-cultured for 16 hours at a 1:1 ratio with T2 cells pulsed with 10-fold dilutions of PRAME_SLLQHLIGL peptide from 1×10⁻⁵ M down to 1×10⁻¹² M. The co-cultures were stained with a cocktail of antibodies to detect the TCR-transduced Jurkat population of cells and any expression of the CD69 activation marker. Samples were acquired using a MACSQuant 10 X Flow Cytometer (Miltenyi Biotec) and the data analysed using FlowJo v10.6.2 (BD). GraphPad Prism v8.2.0 (GraphPad Software, Inc) was used to fit curves to the peptide titration data using a 4-parameter log (agonist) vs. response model of non-linear regression and to derive best-fit values for the EC₅₀; the peptide concentration required to induce half-maximal Jurkat cell activation (% CD69⁺ cells). Results are shown in FIG. 1.

### REFERENCES

1. Jurkat T cells and development of the T-cell receptor signalling paradigm. Abraham R T, Weiss A. 2004, Nat Rev Immunol., pp. 301-8.

### Example 2

### IFNγ ELISPot Assay

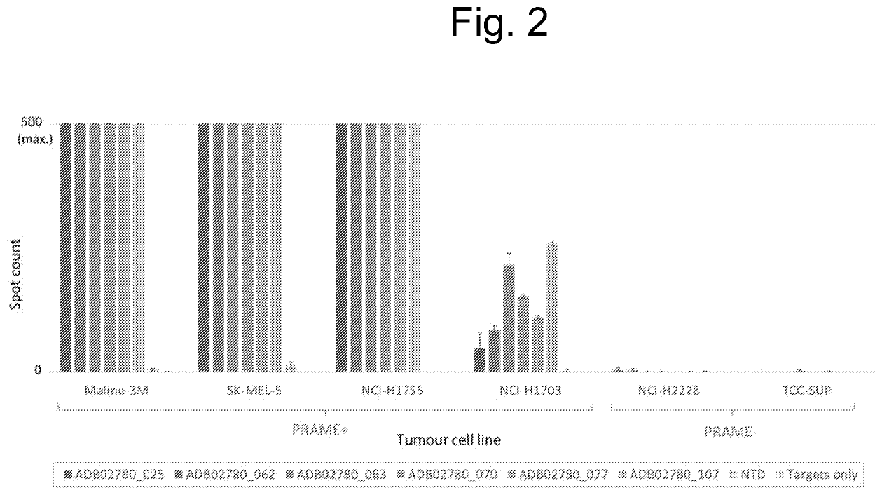

The activation of a panel of TCR-transduced donor T-cells was investigated by measuring local IFNγ secretion captured by antibodies in ELISpot assays. In brief, 96-well ELISpot plates were coated with capture antibody, and incubated for 3 days at 4° C. On the day of assay setup, unbound antibody was removed, and plates blocked with R10 medium for 2 hours at room temperature. Next, 50 000 T-cells were incubated with (or without) 50 000 target cells, both at a concentration of 1×10⁶/mL, for 19 hours at 37° C., in 5% v/v CO₂. Target cells included the PRAME-positive cell lines: Malme-3M and SK-Mel-5 (melanoma); NCI-H1755 and NCI-H1703 (lung); and 2 PRAME-negative (or negligible) tumour cell lines: NCI-H2228 (lung), TCC-SUP (bladder). As controls, target cells without T-cells were monitored, along with T-cells alone (no targets). Plates were developed by washing once in a plate washer, and detection antibody added to each well. After 2 hours of incubation at room temperature, the plates were washed again, and streptavidin-HRP was added to each well. After a further 1-hour incubation at room temperature, the plates were washed a final time. AEC (3-Amino-9-ethylcarbazole) chromogenic substrate was added to each well, and once spots became detectable, plates were washed with water to stop the reaction. Plates were then dried and signals read using a CTL Analyser plate reader (Series 6 Ultra V), and analysed with ImmunoCapture 6.5.8. software. Results are shown in FIG. 2.

### Example 3

### 2D Killing Experiments

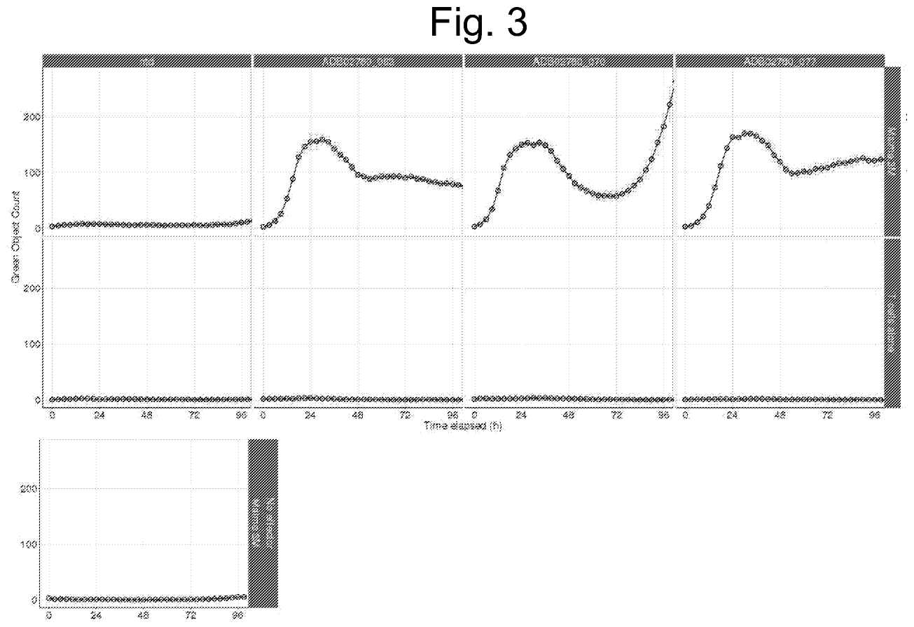

TCR T-cell cytotoxicity towards target cells in co-culture was evaluated by measuring the number of target cells undergoing apoptosis over time using the IncuCyte ZOOM live cell imaging system (Sartorius). Malme-3M (human melanoma) target cells were seeded at 20 000 cells/well in a 96-well plate the day before addition of 50 000 T-cells/well. Target cell apoptosis induced by activated T-cells was quantified using IncuCyte Caspase-3/7 Green Dye (Sartorius) to measure its green fluorescence emitted after cleavage by caspases 3/7 released during apoptosis. Non-transduced T-cells were included as a control for background cell death levels, and Malme-3M target cells alone (no T-cells) were similarly monitored. Image acquisition and analysis were performed using the IncuCyte ZOOM GUI software version 2018A (Sartorius). Apoptotic (green fluorescent) T-cells were gated out by size-exclusion during image analysis. After T-cell addition, plate wells were imaged every 3 hours for approximately 7 days. Data plots show the count of green, fluorescent objects (apoptotic cells)/mm² over time, in hours (FIG. 3).

### Example 4

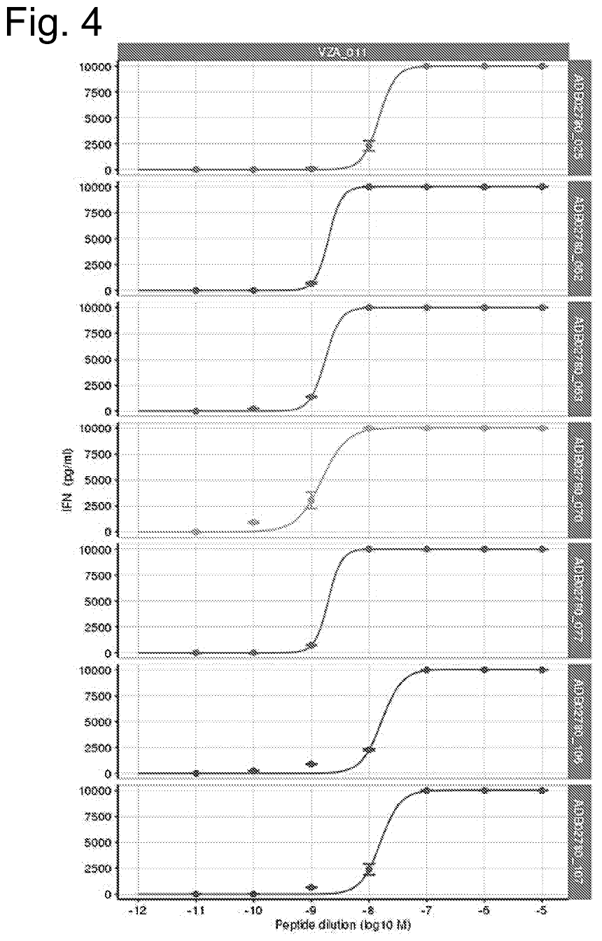

Studies were conducted to further assess the functional avidity and specificity of the potency-enhanced engineered TCRs. To evaluate potency of the TCRs against PRAME-expressing target cells, HLA-A*02 positive, PRAME negative T2 target cells were pulsed with 10-fold dilutions (from 1×10⁻⁵ M to 1×10⁻¹¹ M) of PRAME peptide SLLQHLIGL (SEQ ID NO: 1). Primary T cells were transduced with one of the engineered TCRs provided herein, and co-cultured for 48 hours at a 1:1 ratio with the T2 target cells. Potency was measured by IFNγ supernatant ELISA. Results are provided in FIG. 4 and Table 2. EC₅₀ values are shown in Table 2, and indicate the peptide concentration required to induce half-maximal T-cell activation with values ranging from 10-8.8 M to 10-7.8 M across the panel of TCRs.

TABLE 2
EC₅₀ values

| TCR | EC₅₀ |
|---|---|
| ADB02780_25 | −7.8 |
| ADB02780_62 | −8.7 |
| ADB02780_63 | −8.8 |
| ADB02780_70 | −8.8 |
| ADB02780_77 | −8.7 |
| ADB02780_106 | −7.8 |
| ADB02780_107 | −7.8 |

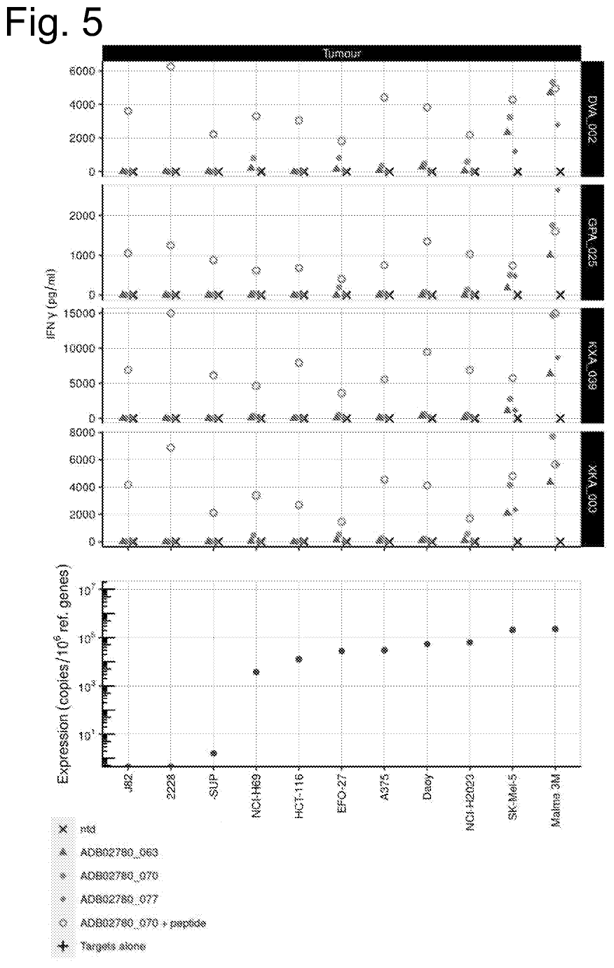

To evaluate the specificity for PRAME, the potency of the enhanced TCRs toward cell lines with various levels of PRAME expression was investigated using IFNγ supernatant ELISA. The results are provided in FIG. 5. T cells from 4 different donors were transduced with an engineered TCR, and incubated with the tumour cell lines shown below the bottom panel, which are ranked by PRAME transcript level (as determined by qPCR) shown in ascending order from left to right.

In general, as shown in FIG. 5, T-cell responses were strongest in response to tumour cell lines with the highest levels of PRAME expression, regardless of the engineered TCR they were transduced with, although potencies varied among TCRs. The 3 cell lines with PRAME expression ranging from negligible to undetectable (<1000 transcript copies/10⁶ reference genes) were not recognised by any of the candidate TCRs in the absence of peptide. All cell lines triggered activation of T-cells transduced to express the ADB02780_070 TCR in the presence of PRAME_SLLQHLIGL peptide (positive control).

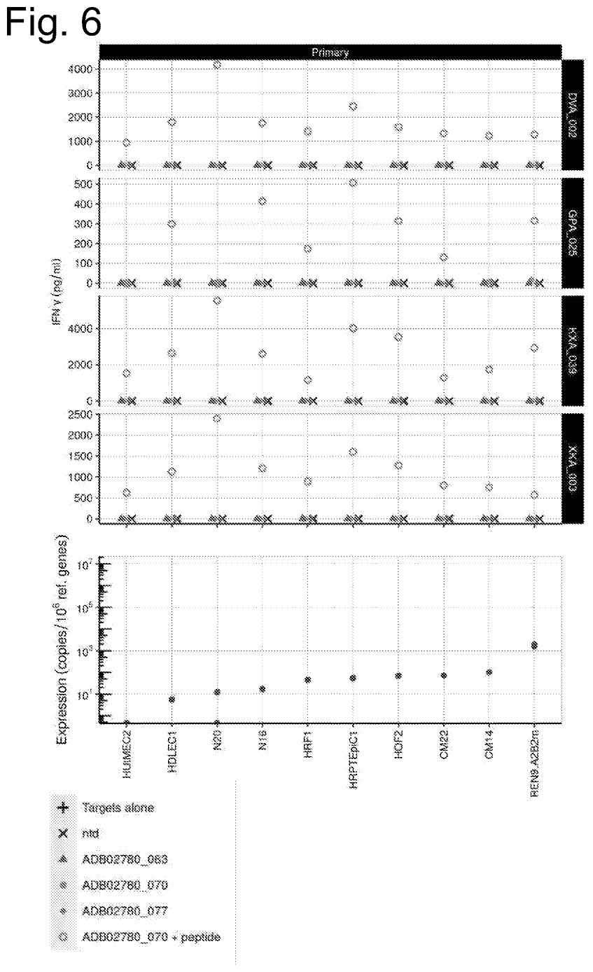

To further demonstrate specificity of the engineered TCRs, T cells from the 4 different donors were transduced with an engineered TCR, and tested against antigen-negative primary cell lines using IFNγ supernatant ELISA. The results are provided in FIG. 6. The transcript level (as measured by qPCR) of the different primary cell lines is shown in the bottom panel. As shown in the top 4 panels, all targets were capable of presenting antigen, as evidenced by ADB02780_070 TCR activation in the presence of exogenous PRAME_SLLQHLIGL peptide (open circles, positive control). However, no meaningful T-cell responses were observed towards any of the PRAME negative primary cell lines in the absence of PRAME_SLLQHLIGL peptide, thus demonstrating the specificity of the engineered TCRs for the PRAME antigen.

1. A T cell receptor (TCR) that binds to a complex comprising SEQ ID NO: 1 and HLA-A*02, and comprises:
(a) a TCR alpha chain variable domain that comprises an amino acid sequence that has at least 50% sequence identity to SEQ ID NO: 17; and
(b) a TCR beta chain variable domain that comprises an amino acid sequence that has at least 50% sequence identity to SEQ ID NO: 29.

2. The TCR of claim 1, wherein:
(a) the TCR alpha chain variable domain comprises an amino acid sequence that has at least 60%, at least 70%, at least 75%, at least 80%, at least 90%, or at least 95% sequence identity to SEQ ID NO: 17; and/or
(b) the TCR beta chain variable domain comprises an amino acid sequence that has at least 60%, at least 70%, at least 75%, at least 80%, at least 90%, or at least 95% sequence identity to SEQ ID NO: 29.

3. The TCR of claim 1 or 2, wherein:
(i) the TCR alpha chain variable domain comprises a CDR1 that comprises (1) the sequence of SEQ ID NO: 8 or (2) an amino acid sequence that comprises one, two or three amino acid insertions, deletions or substitutions relative to the sequence of SEQ ID NO: 8;
(ii) the TCR alpha chain variable domain comprises a CDR2 that comprises (1) the sequence of SEQ ID NO: 15 or (2) an amino acid sequence that comprises one, two or three amino acid insertions, deletions or substitutions relative to the sequence of SEQ ID NO: 15;
(iii) the TCR alpha chain variable domain comprises a CDR3 that comprises (1) the sequence of SEQ ID NO: 16 or (2) an amino acid sequence that comprises one, two or three amino acid insertions, deletions or substitutions relative to the sequence of SEQ ID NO: 16;
(iv) the TCR beta chain variable domain comprises a CDR1 that comprises (1) the sequence of SEQ ID NO: 24 or (2) an amino acid sequence that comprises one, two or three amino acid insertions, deletions or substitutions relative to the sequence of SEQ ID NO: 24;
(v) the TCR beta chain variable domain comprises a CDR2 that comprises (1) the sequence of SEQ ID NO: 25 or (2) an amino acid sequence that comprises one, two or three amino acid insertions, deletions or substitutions relative to the sequence of SEQ ID NO: 25; and/or
(vi) the TCR beta chain variable domain comprises a CDR3 that comprises (1) the sequence of SEQ ID NO: 28 or (2) an amino acid sequence that comprises one, two or three amino acid insertions, deletions or substitutions relative to the sequence of SEQ ID NO: 28.

4. The TCR of any one of the preceding claims, wherein:
(i) the TCR alpha chain variable domain comprises a CDR1 that comprises SEQ ID NO: 8, 9, 10, 11, 12, 13 or 14;
(ii) the TCR alpha chain variable domain comprises a CDR2 that comprises SEQ ID NO: 15;
(iii) the TCR alpha chain variable domain comprises a CDR3 that comprises SEQ ID NO: 16;
(iv) the TCR beta chain variable domain comprises a CDR1 that comprises SEQ ID NO: 24;
(v) the TCR beta chain variable domain comprises a CDR2 that comprises SEQ ID NO: 25, 26 or 27; and/or
(vi) the TCR beta chain variable domain comprises a CDR3 that comprises SEQ ID NO: 28.

5. The TCR of any one of the preceding claims, wherein the TCR alpha chain variable domain comprises:
(i) a CDR1 that comprises SEQ ID NO: 8, a CDR2 that comprises SEQ ID NO: 15, and a CDR3 that comprises SEQ ID NO: 16;
(ii) a CDR1 that comprises SEQ ID NO: 9, a CDR2 that comprises SEQ ID NO: 15, and a CDR3 that comprises SEQ ID NO: 16;
(iii) a CDR1 that comprises SEQ ID NO: 10, a CDR2 that comprises SEQ ID NO: 15, and a CDR3 that comprises SEQ ID NO: 16;
(iv) a CDR1 that comprises SEQ ID NO: 11, a CDR2 that comprises SEQ ID NO: 15, and a CDR3 that comprises SEQ ID NO: 16;
(v) a CDR1 that comprises SEQ ID NO: 12, a CDR2 that comprises SEQ ID NO: 15, and a CDR3 that comprises SEQ ID NO: 16;
(vi) a CDR1 that comprises SEQ ID NO: 13, a CDR2 that comprises SEQ ID NO: 15, and a CDR3 that comprises SEQ ID NO: 16; or
(vii) a CDR1 that comprises SEQ ID NO: 14, a CDR2 that comprises SEQ ID NO: 15, and a CDR3 that comprises SEQ ID NO: 16.

6. The TCR of any one of the preceding claims, wherein the TCR alpha chain variable domain comprises SEQ ID NO: 17, 18, 19, 20, 21, 22 or 23.

7. The TCR of any one of the preceding claims, wherein the TCR beta chain variable domain comprises:
(i) a CDR1 that comprises SEQ ID NO: 24, a CDR2 that comprises SEQ ID NO: 25, and a CDR3 that comprises SEQ ID NO: 28;
(ii) a CDR1 that comprises SEQ ID NO: 24, a CDR2 that comprises SEQ ID NO: 26, and a CDR3 that comprises SEQ ID NO: 28; or
(iii) a CDR1 that comprises SEQ ID NO: 24, a CDR2 that comprises SEQ ID NO: 27, and a CDR3 that comprises SEQ ID NO: 28.

8. The TCR of any one of the preceding claims, wherein the TCR beta chain variable domain comprises SEQ ID NO: 29, 20 or 31.

9. The TCR of any one of the preceding claims, wherein:
(i) the TCR alpha chain variable domain comprises SEQ ID NO: 17 and the TCR beta chain variable domain comprises SEQ ID NO: 29;
(ii) the TCR alpha chain variable domain comprises SEQ ID NO: 17 and the TCR beta chain variable domain comprises SEQ ID NO: 30;
(iii) the TCR alpha chain variable domain comprises SEQ ID NO: 17 and the TCR beta chain variable domain comprises SEQ ID NO: 31;
(iv) the TCR alpha chain variable domain comprises SEQ ID NO: 18 and the TCR beta chain variable domain comprises SEQ ID NO: 31;
(v) the TCR alpha chain variable domain comprises SEQ ID NO: 19 and the TCR beta chain variable domain comprises SEQ ID NO: 31;
(vi) the TCR alpha chain variable domain comprises SEQ ID NO: 20 and the TCR beta chain variable domain comprises SEQ ID NO: 31;
(vii) the TCR alpha chain variable domain comprises SEQ ID NO: 21 and the TCR beta chain variable domain comprises SEQ ID NO: 31;
(viii) the TCR alpha chain variable domain comprises SEQ ID NO: 22 and the TCR beta chain variable domain comprises SEQ ID NO: 31; or
(ix) the TCR alpha chain variable domain comprises SEQ ID NO: 23 and the TCR beta chain variable domain comprises SEQ ID NO: 31.

10. The TCR of any one of the preceding claims, wherein the TCR comprises a TCR alpha chain constant domain and/or a TCR beta chain constant domain.

11. The TCR of any one of the preceding claims, wherein the TCR is an alpha-beta heterodimer comprising:
(a) a TCR alpha chain comprising (i) the TCR alpha chain variable domain and (ii) a TCR alpha chain constant domain; and
(b) a TCR beta chain comprising (i) the TCR beta chain variable domain and (ii) a TCR beta chain constant domain.

12. The TCR of any one of the preceding claims, wherein: the TCR is a single chain TCR of the structure Vα-L-Vβ, Vβ-L-Vα, Vα-Cα-L-Vβ, or Vα-L-Vβ-Cβ; and wherein Vα is the TCR alpha chain variable domain, Vβ is the TCR beta chain variable domain, Cα is TCR alpha chain constant domain, Cβ is a TCR beta chain constant domain, and L is a linker sequence.

13. The TCR of claim 12, wherein the TCR is soluble.

14. The TCR of any one of claims 10 to 13, wherein:
(a) the TCR alpha chain constant domain comprises a TRAC constant domain sequence, optionally wherein the TRAC constant domain sequence comprises SEQ ID NO: 2, SEQ ID NO: 3, or SEQ ID NO: 4; and/or
(b) the TCR beta chain constant domain comprises a TRBC2 constant domain sequence or a TRBC1/2 splice constant domain sequence; optionally wherein the TCR beta chain constant domain sequence comprises SEQ ID NO: 5, SEQ ID NO: 6, or SEQ ID NO: 7.

15. A complex comprising (a) a TCR of any one of the preceding claims, and (b) a detectable label, a therapeutic agent or a pharmacokinetic modifying moiety.

16. A nucleic acid encoding:
(a) a TCR of any one of claims 1 to 14;
(b) a TCR alpha chain variable domain as defined in any one of claims 1 to 14, and optionally a TCR alpha chain constant domain; or
(c) a TCR beta chain variable domain as defined in any one of claims 1 to 14, and optionally a TCR beta chain constant domain.

17. The nucleic acid of claim 16, wherein the nucleic acid encodes a TCR of any one of claims 1 to 14 and:
(a) the nucleic acid encodes the TCR in a single open reading frame; or
(b) the nucleic acid comprises (i) a first open reading frame encoding a TCR alpha chain comprising the TCR alpha chain variable domain and optionally a TCR alpha chain constant domain, and (ii) a second open reading frame encoding a TCR beta chain comprising the TCR beta chain variable domain and optionally a TCR beta chain constant domain.

18. A vector comprising the nucleic acid of claim 16 or 17.

19. A virus particle comprising the nucleic acid of claim 16 or 17, or the vector of claim 18.

20. A cell that:
(a) expresses a TCR of any one of claims 1 to 14;
(b) comprises at least one nucleic acid of claim 16 or claim 17; optionally wherein the cell comprises (i) a first nucleic acid of claim 16 that encodes a TCR alpha chain variable domain as defined in any one of claims 1 to 14 and optionally a TCR alpha chain constant domain, and (ii) a second nucleic acid of claim 16 that encodes a TCR beta chain variable domain as defined in any one of claims 1 to 14 and optionally a TCR beta chain constant domain; or
(c) comprises at least one vector of claim 18; optionally wherein the cell comprises (i) a first vector that comprises a nucleic acid of claim 16 that encodes a TCR alpha chain variable domain as defined in any one of claims 1 to 14 and optionally a TCR alpha chain constant domain, and (ii) a second vector that comprises a nucleic acid of claim 16 that encodes a TCR beta chain variable domain as defined in any one of claims 1 to 14 and optionally a TCR beta chain constant domain.

21. The cell of claim 20, wherein the cell is:
(a) an isolated cell or a non-naturally occurring cell; and/or
(b) a T cell.

22. The cell of claim 20 or 21, wherein the cell is a T cell that further comprises one or more exogenous or recombinant co-stimulatory ligands.

23. A pharmaceutical composition comprising (i) a TCR of any one of claims 1 to 14, a complex of claim 15, a nucleic acid of claim 16 or 17, or a cell of any one of claims 20 to 22, and (ii) one or more pharmaceutically acceptable carriers or excipients.

24. The TCR of any one of claims 1 to 14, nucleic acid of claim 16 or 17, cell of any one of claims 20 to 22, or pharmaceutical composition of claim 23 for use in a method of treating a disease in an individual, the method comprising administering the TCR, nucleic acid, cell or pharmaceutical composition to the individual.

25. A method of treating a disease in an individual, comprising administering to the individual the TCR of any one of claims 1 to 14, nucleic acid of claim 16 or 17, cell of any one of claims 20 to 22, or pharmaceutical composition of claim 23.

26. The TCR, nucleic acid, cell or pharmaceutical composition for use of claim 24, or the method of claim 25, wherein the disease is:
(a) cancer;
(b) a solid tumour; or
(c) selected from melanoma, leukaemia, lymphoma, lung cancer, breast cancer, ovarian cancer, endometrial cancer, oesophageal cancer, bladder cancer and head and neck cancers, optionally wherein (i) the lung cancer is non-small cell lung cancer or small cell lung cancer, or (ii) the breast cancer is triple negative breast cancer.

27. The TCR, nucleic acid, cell or pharmaceutical composition for use of claim 24 or 26, or the method of claim 25 or 26, wherein the method comprises administering a further therapeutic agent to the individual.

28. A method of making one or more cell of any one of claims 20 to 22 comprising:
(i) introducing to a cell at least one nucleic acid of claim 16 or claim 17 or at least one vector of claim 18; and then optionally
(ii) culturing the cell such that the cell proliferates to form a population.

29. The method of claim 28, wherein the at least one nucleic acid or at least one vector is comprised within a virus particle.


SEQUENCE LISTING (39 sequences)

SEQ ID NO: 1 - protein, 9 aa, synthetic construct
SLLQHLIGL

SEQ ID NO: 2 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - protein, 141 aa, synthetic construct
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 94 aa, synthetic construct
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 5 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 6 - protein, 179 aa, synthetic construct
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - protein, 130 aa, synthetic construct
EDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 8 - protein, 5 aa, synthetic construct
SIFNT

SEQ ID NO: 9 - protein, 5 aa, synthetic construct
SIFLT

SEQ ID NO: 10 - protein, 5 aa, synthetic construct
SIFET

SEQ ID NO: 11 - protein, 5 aa, synthetic construct
SIFVT

SEQ ID NO: 12 - protein, 5 aa, synthetic construct
SIFHT

SEQ ID NO: 13 - protein, 5 aa, synthetic construct
SIFAT

SEQ ID NO: 14 - protein, 5 aa, synthetic construct
SIFMT

SEQ ID NO: 15 - protein, 7 aa, synthetic construct
LYKAGEL

SEQ ID NO: 16 - protein, 13 aa, synthetic construct
CAGQASGTYKYIF

SEQ ID NO: 17 - protein, 111 aa, synthetic construct
GQQLNQSPQSMFIQEGEDVSMNCTSSSIFNTWLWYKQDPGEGPVLLIALYKAGELTSNGRLTAQFGITRKDSFLNISASIPSDVGIYFCAGQASGTYKYIFGTGTRLKVLA

SEQ ID NO: 18 - protein, 111 aa, synthetic construct
GQQLNQSPQSMFIQEGEDVSMNCTSSSIFLTWLWYKQDPGEGPVLLIALYKAGELTSNGRLTAQFGITRKDSFLNISASIPSDVGIYFCAGQASGTYKYIFGTGTRLKVLA

SEQ ID NO: 19 - protein, 111 aa, synthetic construct
GQQLNQSPQSMFIQEGEDVSMNCTSSSIFETWLWYKQDPGEGPVLLIALYKAGELTSNGRLTAQFGITRKDSFLNISASIPSDVGIYFCAGQASGTYKYIFGTGTRLKVLA

SEQ ID NO: 20 - protein, 111 aa, synthetic construct
GQQLNQSPQSMFIQEGEDVSMNCTSSSIFVTWLWYKQDPGEGPVLLIALYKAGELTSNGRLTAQFGITRKDSFLNISASIPSDVGIYFCAGQASGTYKYIFGTGTRLKVLA

SEQ ID NO: 21 - protein, 111 aa, synthetic construct
GQQLNQSPQSMFIQEGEDVSMNCTSSSIFHTWLWYKQDPGEGPVLLIALYKAGELTSNGRLTAQFGITRKDSFLNISASIPSDVGIYFCAGQASGTYKYIFGTGTRLKVLA

SEQ ID NO: 22 - protein, 111 aa, synthetic construct
GQQLNQSPQSMFIQEGEDVSMNCTSSSIFATWLWYKQDPGEGPVLLIALYKAGELTSNGRLTAQFGITRKDSFLNISASIPSDVGIYFCAGQASGTYKYIFGTGTRLKVLA

SEQ ID NO: 23 - protein, 111 aa, synthetic construct
GQQLNQSPQSMFIQEGEDVSMNCTSSSIFMTWLWYKQDPGEGPVLLIALYKAGELTSNGRLTAQFGITRKDSFLNISASIPSDVGIYFCAGQASGTYKYIFGTGTRLKVLA

SEQ ID NO: 24 - protein, 5 aa, synthetic construct
MNHEY

SEQ ID NO: 25 - protein, 6 aa, synthetic construct
SMNVEV

SEQ ID NO: 26 - protein, 6 aa, synthetic construct
SLNVEV

SEQ ID NO: 27 - protein, 6 aa, synthetic construct
SINVEV

SEQ ID NO: 28 - protein, 13 aa, synthetic construct
CASSHGTSGEQYF

SEQ ID NO: 29 - protein, 112 aa, synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSHGTSGEQYFGPGTRLTVT

SEQ ID NO: 30 - protein, 112 aa, synthetic construct
EAQVTQNPRYLITVTGKKLTVTCSQNMNHEYMSWYRQDPGLGLRQIYYSLNVEVTDKGDVPEGYKVSRKEKRNFPLILESPSPNQTSLYFCASSHGTSGEQYFGPGTRLTVT

SEQ ID NO: 31 - protein, 112 aa, synthetic construct
EAQVTQNPRYLITVTGKKLTVTCSQNMNHEYMSWYRQDPGLGLRQIYYSINVEVTDKGDVPEGYKVSRKEKRNFPLILESPSPNQTSLYFCASSHGTSGEQYFGPGTRLTVT

SEQ ID NO: 32 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 93 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 35 - protein, 178 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 36 - protein, 178 aa, synthetic construct
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - protein, 129 aa, synthetic construct
DLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 38 - protein, 18 aa, synthetic construct
MLLEHLLIILWMQLTWVS

SEQ ID NO: 39 - protein, 19 aa, synthetic construct
MGPQLLGYVVLCLLGAGPL